(19) United States
(12) Patent Application Publication
(10) Pub. No.: US 2026/0306183 A1
(43) Pub. Date: Oct. 8, 2026

(54) TISSUE RESECTION PROGRESS INDICATOR

(71) Applicant: IQ Surgical, Inc., Dover, DE (US)

(72) Inventors: Richard W. Timm, Dover, DE (US); Vivek P. Buch, Dover, DE (US); Jay J. Park, Dover, DE (US)

(21) Appl. No.: 19/642,306

(22) Filed: Apr. 8, 2026

Related U.S. Application Data
(60) Provisional application No. 63/785,216, filed on Apr. 8, 2025.
(60) Provisional application No. 63/809,918, filed on May 21, 2025.

(51) Int. Cl. A61B 34/10; A61B 34/00; G06T 7/00; G06T 15/08; G06T 17/20

(52) U.S. Cl. CPC A61B 34/10; A61B 34/25; G06T 7/0012; G06T 15/08; G06T 17/20; A61B 2034/101; G06T 2200/24; G06T 2207/10012; G06T 2207/30096; G06T 2207/30168; G06T 2210/41

(57) ABSTRACT

Provided is a method including obtaining a first volumetric image dataset of an anatomical region of a patient, obtaining a planned resection model associated with the first volumetric image dataset, the planned resection model indicating a surgeon-planned resection region within the anatomical region, receiving optical image data of the anatomical region captured during a surgical procedure, receiving a signal indicating that a resection progress measurement is to be initiated, constructing a three-dimensional mesh reconstruction of tissue visible in the anatomical region during the surgical procedure, determining an updated anatomical model using the first volumetric image dataset, the three-dimensional mesh reconstruction, and a model configured to update the first volumetric image dataset to account for intraoperative tissue displacement in the anatomical region, comparing the updated anatomical model with the planned resection model to determine a resection progress indication relative to the surgeon-planned resection region, and presenting an output indicating resection progress.

### CROSS-REFERENCE TO RELATED APPLICATIONS

This patent claims the benefit of U.S. Provisional Patent Application 63/785,216, filed Apr. 8, 2025, titled TISSUE RESECTION PROGRESS INDICATOR and U.S. Provisional Patent Application 63/809,918, filed May 21, 2025, titled ADAPTIVE MULTI-MODALITY SURGICAL RESECTION TRACKER. The entire content of each afore-listed earlier-filed application is hereby incorporated by reference for all purposes.

### BACKGROUND

### 1. Field

The present disclosure relates generally to computer-implemented tools to assist healthcare providers and, more specifically, to a tissue resection progress indicator.

### 2. Description of the Related Art

A surgeon might need to resect brain tissue to remove a tumor, alleviate seizures caused by epilepsy, or relieve pressure from swelling, bleeding, or other abnormalities. This procedure can be necessary when the affected tissue is causing significant neurological symptoms or threatening the patient's life. By removing the problematic area, surgeons aim to preserve as much healthy brain function as possible while addressing the underlying condition to improve the patient's quality of life or survival.

When removing brain tumors, surgeons walk a tightrope between taking out enough tissue to eliminate as much of the tumor as possible and preserving the surrounding healthy brain tissue that controls critical functions like speech, movement, and memory. Removing too little increases the risk that the tumor will grow back or continue to cause symptoms but removing too much can cause permanent neurological damage. To strike the right balance, surgeons use advanced imaging and mapping techniques to precisely identify and avoid essential areas of the brain while still achieving the best possible outcome.

Similar issues arise in other forms of surgery in which material is removed while wishing to preserve functionality of adjacent tissues.

### SUMMARY

The following is a non-exhaustive listing of some aspects of the present techniques. These and other aspects are described in the following disclosure.

Some aspects include a process, including: initiating 3D reconstruction on intraoperatively captured images and checking quality of data capture; performing 3D reconstruction and generating surface position information of surgical scene; identifying resection regions of intraoperative captured images where tissue has been removed; performing volumetric analysis on the resection regions; defining resection target tissue from preoperative image; and comparing volumetric analysis of resection regions to volumetric analysis of resection target tissue.

Some aspects include a method including obtaining a first volumetric image dataset of an anatomical region of a patient, obtaining a planned resection model associated with the first volumetric image dataset, the planned resection model indicating a surgeon-planned resection region within the anatomical region, receiving optical image data of the anatomical region captured during a surgical procedure, receiving a signal indicating that a resection progress measurement is to be initiated, constructing a three-dimensional mesh reconstruction of tissue visible in the anatomical region during the surgical procedure, determining an updated anatomical model using the first volumetric image dataset, the three-dimensional mesh reconstruction, and a model configured to update the first volumetric image dataset to account for intraoperative tissue displacement in the anatomical region, comparing the updated anatomical model with the planned resection model to determine a resection progress indication relative to the surgeon-planned resection region, and presenting an output indicating resection progress.

Some aspects include a tangible, non-transitory, machine-readable medium storing instructions that when executed by a data processing apparatus cause the data processing apparatus to perform operations including the above-mentioned process.

Some aspects include a system, including: one or more processors; and memory storing instructions that when executed by the processors cause the processors to effectuate operations of the above-mentioned process.

### BRIEF DESCRIPTION OF THE DRAWINGS

The above-mentioned aspects and other aspects of the present techniques will be better understood when the present application is read in view of the following figures in which like numbers indicate similar or identical elements:

FIG. 1 illustrates example rendered views associated with a planned resection region and example progress information relative to the planned resection region.

FIG. 2 illustrates an example operating-room environment in which optical image data is captured and processed to determine resection progress.

FIG. 3 illustrates example states of a surgical scene that may be evaluated to determine whether resection-progress analysis is to be initiated.

FIG. 4 illustrates example initiation conditions for three-dimensional reconstruction and volumetric analysis.

FIG. 5 illustrates an example process for evaluating initiation conditions and measurement-quality conditions before reconstruction and volumetric analysis.

FIG. 6 illustrates example comparisons of optical image data, depth information generated using different reconstruction approaches, and corresponding contour representations of a resection cavity.

FIG. 7 illustrates example determination of a target volume or planned resection region from volumetric imaging data.

FIG. 8 illustrates an example comparison of a resection mask to a surgical plan.

FIG. 9 illustrates an example process for determining resection progress using volumetric image data and intraoperative optical image data.

FIG. 10 illustrates an example computer system by which the present techniques may be implemented.

While the present techniques are susceptible to various modifications and alternative forms, specific embodiments thereof are shown by way of example in the drawings and will herein be described in detail. The drawings may not be to scale. It should be understood, however, that the drawings and detailed description thereto are not intended to limit the present techniques to the particular form disclosed, but to the contrary, the intention is to cover all modifications, equivalents, and alternatives falling within the spirit and scope of the present techniques as defined by the appended claims.

### DETAILED DESCRIPTION OF CERTAIN EMBODIMENTS

To mitigate the problems described herein, the inventors had to both invent solutions and, in some cases just as importantly, recognize problems overlooked (or not yet foreseen) by others in the field of computer vision. Indeed, the inventors wish to emphasize the difficulty of recognizing those problems that are nascent and will become much more apparent in the future should trends in industry continue as the inventors expect. Further, because multiple problems are addressed, it should be understood that some embodiments are problem-specific, and not all embodiments address every problem with traditional systems described herein or provide every benefit described herein. That said, improvements that solve various permutations of these problems are described below.

Achieving a “maximum safe resection” of brain tumors is a commonly cited goal to balance the improvement of patient survival versus risking neurological deficit. However, in malignant tumors, the percent of tumor successfully removed is frequently lower than the 80% required to establish prolonged patient survival. And while avoiding neurologic deficit contributes to intentional decision to sometimes leave tumor behind for safety reasons, in many cases, tumor is left behind unintentionally because of the difficult problem of maintaining accurate spatial awareness of the boundaries of 3-dimensional shape of tumor versus healthy brain tissue. When the risk to the patient is too high, either due to immediate intraoperative factors like the risk of severe bleeding, stroke, or death, or due to postoperative deficits due to risk of damaging critical neural structures and eloquent tissue, surgeons may opt for a more conservative resection, leaving behind tumor tissue. In these cases, this judgement is subjective and informed by the surgeon's own understanding of the spatial relationships between anatomic features and weighing the risk versus benefit.

One of the main factors that complicates the problem of spatial awareness is brain shift. While some neuronavigation approaches provide an accurate (e.g., <2 mm error) method of spatially tracking handheld surgical tools and other instruments and registering their actual positions with respect to a patient's pre-operative 3D imaging (MRI, CT), often, the registration only remains accurate with respect to bony landmarks. Soft tissues, which include brain tissue and tumor, deform due to surgical manipulation. Because neuronavigation based solely on pre-operative 3D imaging becomes inaccurate due to increasing brain shift that can reach to 2 cm displacement as compared to preoperative imaging, it no longer helps sufficiently with spatial awareness and discrimination of tumor boundaries, which is not to suggest that this, or any other, approach is disclaimed or disavowed.

Other brain lesions that are treated through resection share the problem of brain shift. In resections of epilepsy lesions, arteriovenous malformations (AVMs), parasitic infections, and other types of lesions, brain shift causes neuronavigation error that forces the neurosurgeon to rely on a combination of their experience and ability to sense tactile feel of tissue and visual discrimination to determine resection margins. In these cases, it is also possible to under-resect the lesion of interest due to the complex spatial awareness problem due to brain shift.

In other parts of the body, it is also important to preserve as much healthy tissue as possible during lesion resections or other types of resections. The challenge with spatial awareness of the tumor within the solid tissue, as well as spatial relationship with other critical structures (e.g., vessels, nerves, ducts, other organs, etc.) is also of high concern during surgery. Notable anatomic examples where tumor resection is critical while preserving otherwise healthy tissue include the breast, lung, and liver.

Surgeon experience greatly informs their own perception and estimate of resection progress. Surgeons, especially ones with less experience, may have a difficult time estimating how much tumor has been removed. Anecdotally according to one senior attending neurosurgeon, surgical residents may report having removed 80+% of tumor, whereas the attending surgeon will observe only 40% removal. Given the goal of maximum safe resection, which achieves oncologic benefit through decellularization, leaving too much remnant tumor will fail to realize the surgical objective.

To mitigate these or other challenges, some embodiments provide objective and quantified information about the progress of the resection as a tool for surgeons to assess their progress.

Some approaches perform modeling and compensating for brain shift, which can give quantified feedback about the progress of the resection. These approaches may be technologically manageable, while still providing meaningful and actionable information to support clinical decision making. One of the difficult challenges cranial neurosurgeons face is when to stop resecting. Informing this decision is their own judgment about the oncologic benefits vs. risk of causing intraoperative complication or postoperative neurological deficit. Having additional objective information, beyond that provided by the afore-mentioned modeling and compensating, to support this decision is expected to result in better oncologic (and other) outcomes.

Some embodiments include an image processing module that quantifies and tracks resection progress at one or more points in the procedure. An example embodiment uses (e.g., can retrofit without supplementing the sensor suite of) existing hardware in the operating room (OR), such as a surgical microscope, neuronavigation system. This has the advantage of minimizing new hardware, which helps to simplify development and reduce cost. For example, some embodiments may operate based solely on image data from one or more optical cameras, such as two optical cameras in a stereo array, such as those integrated into a surgical microscope or neuro-navigation system. Some embodiments may not include an integrated MRI machine, ultrasound, depth camera, or other non-optical techniques that would impede efforts to retrofit existing tooling in the operating room that only includes one or two optical cameras with the resection site in the field of view (e.g., from a static camera pose, or from a camera pose that is static between movements driven by the surgeon).

In some embodiments, other types of systems, sensor suites, and data may be used. While the present disclosure describes the image processing module as a potentially separate computation system, it is easily envisioned that the hardware and software for this module is deeply integrated with either existing surgical microscope systems, or with neuronavigation systems. Examples include MRI systems, LIDAR, tumor margin probes, fluorescence guided surgery, ultrasound, depth cameras, and the like, in some cases in combination. In some embodiments, sensor fusion algorithms may be used with these additional channels of sensing and the other techniques described herein to further improve patient outcomes.

In some embodiments, the image processing module may receive image data (e.g., stereoscopic video or still images) from a microscope, which may capture stereoscopic red-green-blue images to present a 3D view to the surgeon. The image data from the microscope may be used as a (e.g., sole, when combined with pre-operative 3D imaging) basis for 3D reconstruction. However, other wavelengths of electromagnetic radiation, including infrared and ultraviolet may offer additional information content and may be used in some embodiments. Alternatively or additionally, LIDAR systems may be used for depth estimation and 3D reconstruction. While standard surgical microscopes do not contain LIDAR today, a system fitted with LIDAR as a means of obtaining more accurate depth measurements provides a method that could be used in some embodiments.

Alternatively or additionally, the ability to perform intraoperative imaging on soft tissue (CT, MRI) without disrupting the workflow provides another method of quantifying resection progress vs. the target. This is expected to allow another means of reconstruction of the 3D tumor cavity, and comparing the amount of tumor removed against the intended resection.

Computer vision, e.g., with deep learning, is expected to provide accurate 3D reconstruction regardless of optical camera calibration, and without the use of stereo vision methods, in some embodiments.

In some embodiments, neurosurgery will be assisted with a robotic system controlling (e.g., autonomously) the trajectory of resection tools. By planning or tracking the trajectory, boundaries of dissection and resection can be estimated. This would provide another means of calculating resection progress for comparison against the preoperative plan.

In some embodiments, a result of the analysis depicted above may be presented to a surgeon in a user interface, such as on a display screen or augmented reality headset. In the operating room, some embodiments may present an augmented view of the tissue being resected, showing both regions that remain to be resected and regions that are not to be resected, e.g., with highlighting, text labels, blinking, etc., in the UI.

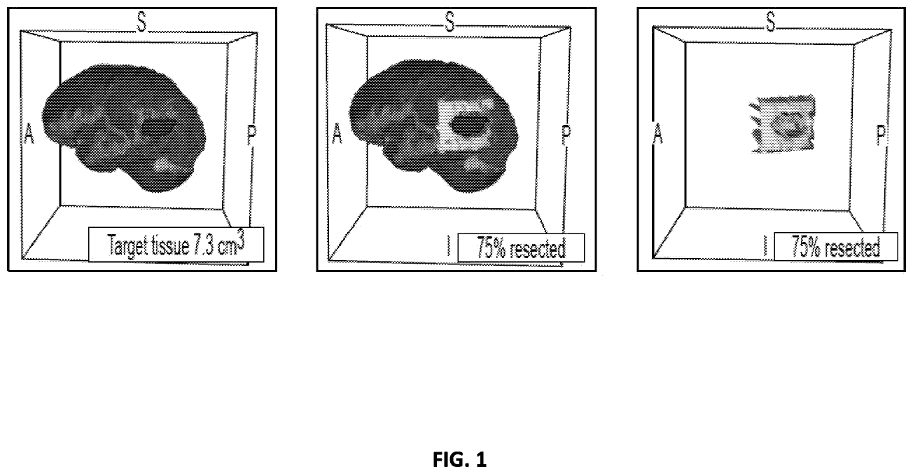

FIG. 1 illustrates example views of a patient-specific volumetric representation in which a planned resection region and resection-progress information are presented relative to an anatomical region. In some embodiments, the displayed content may be generated from preoperative volumetric image data, such as magnetic resonance imaging or computed tomography data, together with intraoperative analysis results derived from optical image data captured during a surgical procedure. The views may present the anatomical region in a rendered three-dimensional perspective, in slice-based views, or both. In some embodiments, the presentation may distinguish tissue included within a planned resection region, tissue already corresponding to a resected region, and tissue remaining within the planned resection region.

FIG. 1 further illustrate that, in some embodiments, resection progress is presented relative to a surgeon-planned target rather than solely as an updated image of anatomy. For example, the displayed information may indicate a portion of the planned resection region that has already been resected, a portion that remains unresected, or both. In some embodiments, the rendered perspective may be selected to correspond generally to a surgical viewpoint, while in other embodiments the information may additionally or alternatively be presented in axial, coronal, sagittal, or other views derived from the volumetric image data. The depicted views are examples, and other visualizations may be used to convey progress relative to a planned resection region.

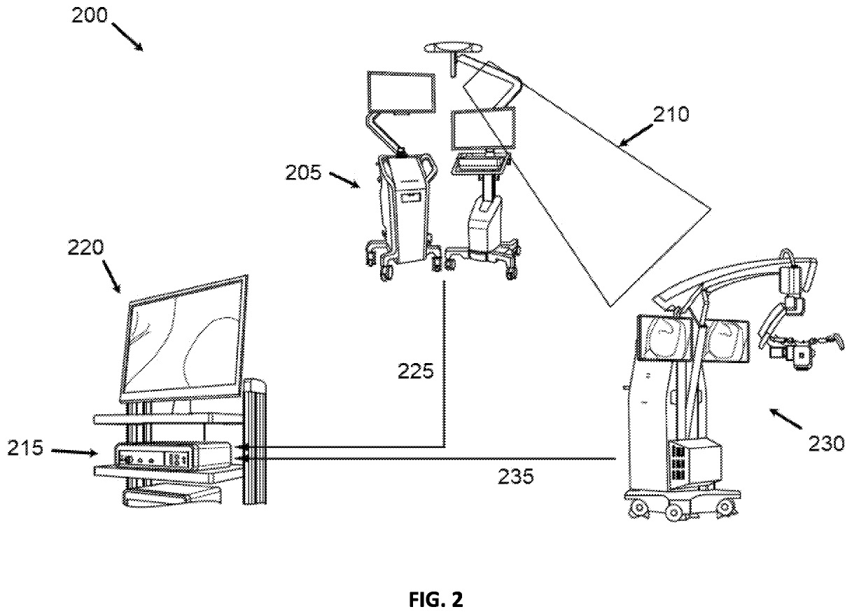

FIG. 2 illustrates an example in-operating-room system 200 for determining and presenting resection-progress information during a surgical procedure. In some embodiments, the system 200 may include a navigation system 205, an image processing module 215, and a monitor 220 for presenting results of analysis. The navigation system 205 may include hardware 210 for patient tracking, image acquisition, or both. The navigation system 205 and the hardware 210 may provide spatial tracking information 225 to the image processing module 215. In some embodiments, the spatial tracking information 225 may indicate positions, orientations, registration information, or other spatial relationships associated with the patient, one or more instruments, one or more imaging devices, or combinations thereof.

In some embodiments, the system 200 may further include optical image acquisition hardware 230, such as a surgical microscope. The optical image acquisition hardware 230 may provide live video and metadata 235 to the image processing module 215. The metadata may include, for example, timing information, imaging parameters, zoom information, focus information, pose information, calibration information, stereoscopic image relationship information, or other information associated with captured optical image data. The image processing module 215 may use the spatial tracking information 225, the live video and metadata 235, or both, to determine resection-progress information and provide output for presentation on the monitor 220.

### Initiation of 3D Reconstruction and Volumetric Analysis

Accurate and clinically-relevant 3D reconstruction of the surgical scene and resection region may occur when the surface of the tissue is measured with less than approximately 4 mm error relative to the actual surface. To achieve this accuracy (or less, or more, in some embodiments), capturing good quality image data is helpful. However, for much of the surgical procedure, obstructions and tissue manipulation result in image data that is unsuitable for accurate 3D reconstruction. Additionally, for phases and steps of the procedure besides the resection, it does not make sense to quantify resection progress. Other factors like camera position may also result in incomplete capture of the surgical scene, or create obstructions that would otherwise prevent accurate measurement. For example, for many tumor resections, and based on surgeon preference, prior to opening the dura mater, the surgical microscope may not even be in a position over the surgical field. It is desirable to not only capture accurate and high-quality data, but also to minimize disruption of surgical workflow for data capture purposes (e.g., by requiring the surgeon to manually label images as suitable or not suitable for use).

In some embodiments, the system automatically detects the optimal measurement conditions for 3D reconstruction and volumetric analysis of the resection cavity from surgical video. Initiation conditions for automatic analysis may include one or more of the conditions listed below.

In some embodiments, the surgical microscope or capture device may be in position to capture images of the relevant portions of the surgical field. However, for many digital microscope systems, the view of the surgeon is different from the digital image outputs. This is because the surgeon sees through the optics of the microscope, perceived as a circular shape, whereas the digital capture corresponds to a cropped portion of the image, often a rectangular shape, which exists entirely within the circle. Therefore, it may not be apparent to the surgeon that the image processing device only sees a portion of what they see. To evaluate this condition, images of the surgical field may be processed with a pre-trained machine learning model performing inference in near-real time (e.g., within less than 2 seconds of image receipt, e.g., within less than 500 ms). Examples of pre-trained ML models include trained convolutional neural networks (CNNs), recurrent neural networks, temporal convolutional neural networks, vision transformers, 3D CNNs, and multimodal models that fuse inputs from multiple sources for temporal classification.

In some embodiments, analysis may be performed during the appropriate phase or step of the procedure in some embodiments. Automatic temporal segmentation of the procedural phase and step may be achieved, in some embodiments, using a pre-trained machine learning model performing inference in near-real time as per above examples. 3D reconstruction in brain tumor surgery is often only relevant during the main resection portion of the procedure, which occurs after completion of the durotomy, and prior to repair of the durotomy. Temporal segmentation in some embodiments can identify this step of resection.

In some embodiments, alternatively or additionally, an initiation condition may be a presence or object detection within the image of the surgical scene or anatomic feature, such as the pia mater. This may be achieved through a pre-trained machine learning method performing inference in near-real time as per above examples.

In some embodiments, once the initiation condition(s) for automated detection of measurement conditions are met, the system may automatically initiate analysis of images and 3D reconstruction.

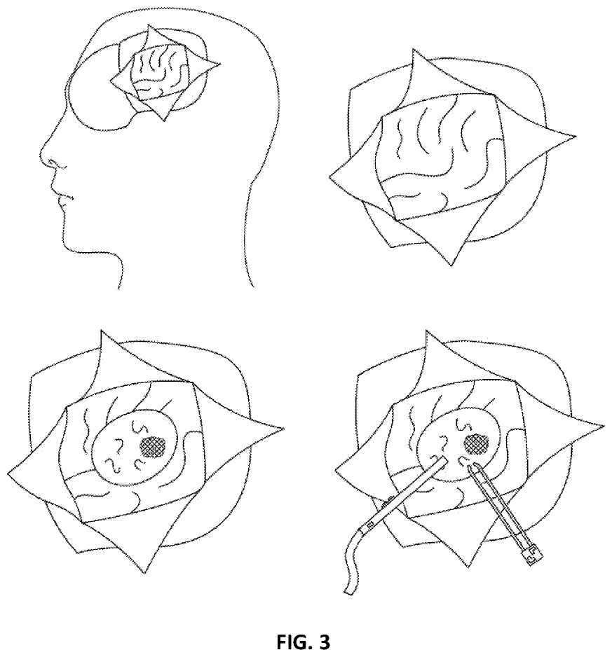

FIG. 3 illustrates example intraoperative optical image states that may be used in determining whether resection-progress analysis is to be initiated. In some embodiments, the depicted image states may correspond to different views of a surgical field captured during a procedure, including views in which relevant anatomy or a resection cavity is visible and views in which one or more instruments are present in the field of view. The image processing module may evaluate such image data to determine whether a portion of the anatomical region associated with a planned resection is sufficiently visible for subsequent processing, whether tissue removal is underway, or whether an image-based trigger condition is satisfied.

In some embodiments, the determination associated with FIG. 3 may be based on one or more image-analysis operations performed on the captured optical image data. For example, the system may process the image data to detect an anatomic feature, detect a surgical instrument, classify a procedural state, or otherwise determine whether the current scene corresponds to a state in which three-dimensional reconstruction or volumetric analysis should proceed. In some embodiments, a triggering instrument may be recognized using object detection, segmentation, fiducial recognition, or other computer-vision processing, and in some cases the image state may be evaluated together with manual or other external trigger inputs. FIG. 3 is illustrative, and other image states and trigger conditions may be used, which is not to imply that any other description is limiting.

As an alternative to, or supplement to, automated initiation of 3D reconstruction and volumetric analysis, this can be initiated by the surgeon manually. The benefit of manual initiation is that, in some embodiments, it engages the surgeon to take proactive steps for better measurement quality, such as zooming the camera out for a wider view of the field and removing instruments or other obstructions from the surgical scene. In some embodiments, one or more of these conditions may need to be met to initiate analysis:
a. Voice command—the system may include a microphone on a handheld probe, or an audio signal input from another device. Voice recognition algorithms may monitor for “wake” words, the detection of which would trigger image capture and analysis. Or speech-to-text models may output text to a transformer that maps utterances to commands.
b. Press of a physical button or touch sensor. A button on a sterile handheld probe connected to the image processing module may trigger initiation of 3D reconstruction. Alternatively, this button may reside on the image processing module itself. Alternatively, this button may reside on the microscope, with button state information communicated either separately from image data, or embedded within the image data.
c. Automatic recognition of a particular instrument brought into the surgical scene. A pre-trained computer vision model may be used to detect a surgical suction device using an object detector, semantic segmentation, or instance segmentation. However, in an alternate embodiment, the instrument is attached to at least one optical fiducial, such as an ArUco marker. Recognition of this tool in the visual field may trigger subsequent steps.
d. Some embodiments may perform gesture recognition on a tool or the surgeon's hand. Upon detecting that the surgeon has performed a gesture within the field of view of the cameras and classifying that gesture as corresponding to a command, some embodiments may initiate subsequent steps.

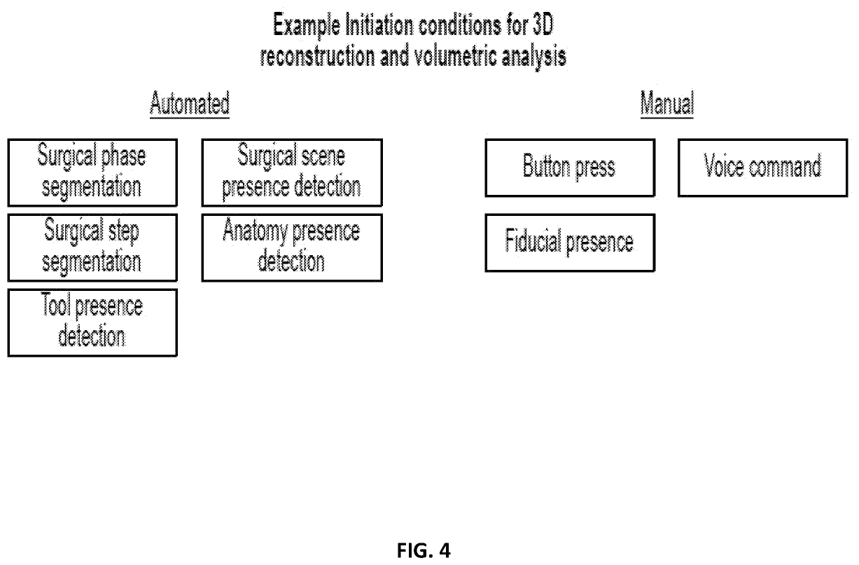

FIG. 4 illustrates example initiation conditions that may be evaluated before three-dimensional reconstruction or volumetric analysis is performed. In some embodiments, the initiation conditions may include conditions indicating that the procedure has advanced to a stage in which tissue removal from the anatomical region is underway, that a surgical microscope or other capture device is positioned to image a relevant portion of the surgical field, or that optical image data includes anatomy associated with the anatomical region. In some embodiments, one or more image-analysis operations may be performed on captured optical image data to determine whether such conditions are satisfied, including procedural-stage classification, anatomic-feature detection, or object detection performed with one or more trained models.

FIG. 4 also shows example command-based or event-based initiation conditions. For example, initiation may occur in response to detection of a triggering instrument in the field of view, receipt of a voice command, receipt of a button press or touch input, or recognition of a gesture associated with a command. Manual initiation may be used alone or together with automatically evaluated initiation conditions. In some embodiments, satisfaction of one or more initiation conditions may cause the system to begin image analysis, three-dimensional reconstruction, or subsequent volumetric processing of a resection region.

### Quality of Image Capture for 3D Reconstruction and Volumetric Analysis

Conditions that, in some embodiments, are expected to improve quality of automatically-initiated 3D reconstruction include the following:
a. There should be no instrument or other artificial obstructions of the cavity, e.g. surgical sponges, cottonoid, suction devices, bipolar forceps.
b. There should be minimal or no pools of blood obstructing visibility of the field
c. Minimal movement of tissue or of the camera itself
d. The entire surgical cavity should be in focus

To ensure quality of acquired images, it may be helpful to quantify the quality of the captured frame(s) before proceeding to subsequent analysis. Failure of acquisition quality to meet defined thresholds may prompt one or more of: warning of capture quality, re-acquisition by the user, and/or display of instructions to the user to facilitate higher quality data capture. Some embodiments may perform one or more of the following:
a. The system may capture multiple images for temporal smoothing of acquisition noise.
b. Detection of artificial obstructions, as well as pools of blood, may be achieved through a pre-trained machine learning model performing inference on one or more captured images. Semantic segmentation masks that identify obstructions may be used to exclude regions of pixels from the 3D reconstruction.
c. Examples of machine learning models include encoder/decoder networks such as U-NetPlus, attention-based methods, and vision transformer models.
d. Detection of tissue movement, or general movement of the image itself may be achieved via optical flow methods applied to sequential frames. When taking multiple images over time (e.g., with video capture), segments of video associated with optical flow vector magnitudes that surpass defined thresholds may be excluded from the analysis. The threshold may include an average magnitude across the entire image, or may be applied to sub-regions of the image to detect more localized movement. Potential optical flow methods include approaches like Lucas-Kanade or deep learning approaches like CoTracker3. Alternatively, object tracking algorithms may be used to quantify the motion of regions of the image, instruments affixed to tissue, fiducials affixed to tissue, markings drawn on anatomy by a surgical pen, or tracking portions of anatomy.
e. (e) Detection of image being out of focus or blurred may be achieved by taking the Laplacian of at least one channel of the image and calculating the variance. If the variance falls below a threshold, for example, 100, the image is deemed blurry. The Laplacian can be calculated by convolving the 3×3 Laplacian kernel over the image, such as the following.

$$\begin{matrix}[ \begin{matrix}0 & 1 & 0 \\ 1 & - 4 & 1 \\ 0 & 1 & 0\end{matrix} ] \\ Laplacian kernel\end{matrix}$$

f. An overall image acquisition quality score may be calculated to determine whether to accept or reject acquired frames. In a preferred embodiment, the acquisition quality score may be a weighted composite of optical flow vector magnitude, variance of the Laplacian, and the percentage of area obstructed by either instruments or blood.

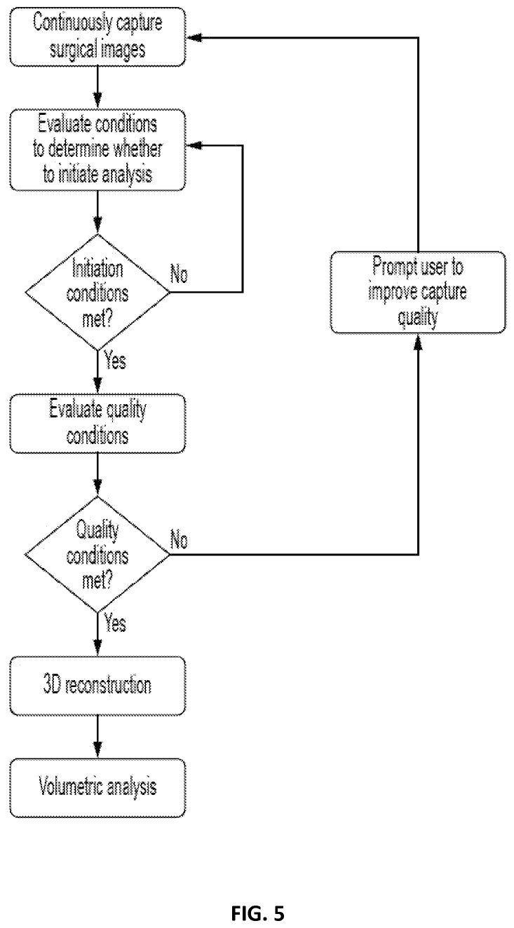

FIG. 5 illustrates an example process for determining whether image data captured during a surgical procedure is suitable for use in resection-progress analysis. In some embodiments, the process may be executed by a computer system like that described below integrated with or communicatively coupled with a surgical microscope or other source of cameras or other sensors. In some embodiments, the process may include receiving optical image data from a surgical microscope or other optical imaging device and evaluating one or more initiation conditions to determine whether subsequent processing is to proceed. The initiation conditions may include, for example, whether tissue removal from an anatomical region is underway, whether a field of view includes a relevant portion of the anatomical region, whether a triggering instrument is present, or whether a manual or other command has been received. When one or more initiation conditions are satisfied, the process may continue to evaluation of one or more measurement-quality conditions for the captured image data.

In some embodiments, the measurement-quality conditions may include whether the captured image data satisfies one or more visibility, focus, motion, or obstruction conditions. For example, the process may evaluate whether relevant anatomy or a resection cavity is sufficiently visible, whether blur or motion is below a threshold, or whether blood, tools, or other structures obscure too much of the field of view. In response to determining that the initiation conditions and the measurement-quality conditions are satisfied, the process may proceed with three-dimensional reconstruction, cavity analysis, volumetric analysis, or other determination of resection-progress information. If one or more conditions are not satisfied, the process may defer or refrain from performing the subsequent analysis until suitable image data is obtained.

In response to classifying an image as being of insufficient quality through the approaches described above, some embodiments may discard that image or downweight that image, or portions thereof classified as below threshold quality, when performing 3D reconstruction. Some embodiments may further determine a number of consecutive images or a number of images within a trailing duration of time that fail to meet quality thresholds. And upon determining that the number exceeds some quality threshold number, some embodiments may emit an alarm or otherwise warn the surgeon that image quality may be impaired. Some embodiments may visually augment a display to identify regions with low image quality.

### 3D Reconstruction of Intraoperatively Acquired Images

Some embodiments use a stereo computer vision method (e.g., rectification+stereo semi-global block matching+disparity calculation and transformation via camera parameters) to calculate a depth map of the surgical scene. The depth map can be converted to a point cloud via transformation using camera calibration parameters, e.g., determining offset of each pixel from the optical center and then dividing by the focal length.

Depth maps attained through non-deep-learning (e.g., classical) computer vision methods can be quantitatively accurate for much of the image, but also may have a degree of sparsity, e.g., a mix of valid and invalid points. Causes of invalid points may include but are not limited to (which is not to suggest other lists are limiting) the camera optics, low-texture surface, occlusion, parallax effects making object surfaces appear different from different angles, and lighting differences, among other challenges that cause failure to accurately calculate disparity, e.g., pixel-to-pixel distance between the same features in the two source images. Invalid points can be identified and excluded in many cases based on pixel depth values that are too high or too low (e.g., above or below threshold). And while excluding invalid points from measurement is one approach, there may be invalid points that are in important areas of measurement. In many instances, these invalid points reside in regions of sharp geometric discontinuity of depth, leading to mismeasurement at edges of surgical resection cavities. Thus, using multiple techniques in combination may be helpful to achieve sufficient accuracy for clinical use.

In some embodiments, use of a secondary computer vision method for depth map completion can “fill in” regions of invalid points and serve as an independent quality check of the rest of the image. In some embodiments, the secondary computer vision method may output values with good relative accuracy but not absolute accuracy, its output may be scaled using the classical model output by choosing two or more points, which may be normalized, and/or interpolated.

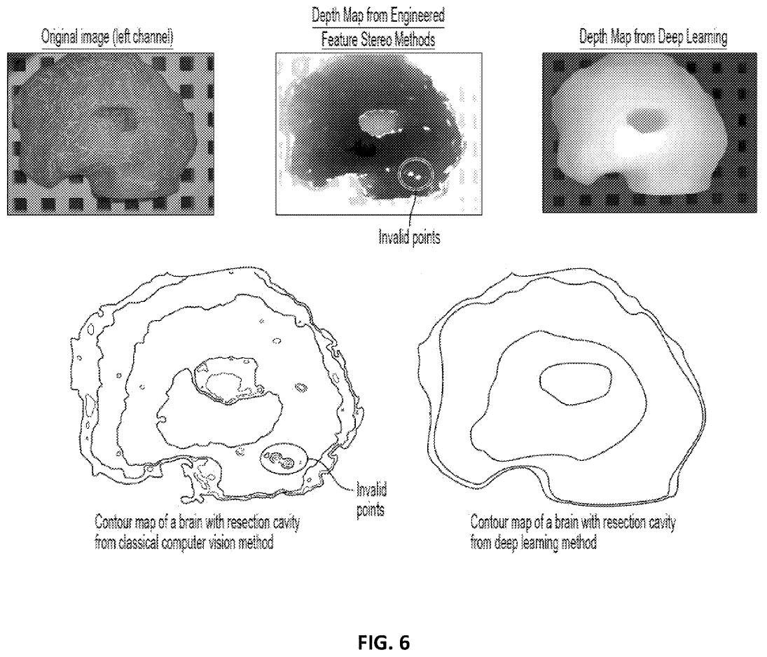

FIG. 6 shows example data associated with determination of depth information and surface information from optical image data captured during a surgical procedure. In some embodiments, the figure may include an example optical image of an anatomical region, a first depth representation generated using a stereo-based reconstruction process, and a second depth representation generated using a learned depth-completion or depth-estimation process. In some embodiments, the stereo-based reconstruction process may produce invalid or sparse depth values in portions of the field of view, and the learned process may be used to supplement or replace missing depth information for those portions. The resulting depth information may be used to generate a three-dimensional representation of a surgical scene, including a point cloud, surface representation, contour representation, or other geometry associated with a resection cavity or surrounding anatomy.

FIG. 6 further illustrate that, in some embodiments, different reconstruction approaches may be compared based on the resulting geometric representation of the anatomical region. For example, contour lines, cavity boundaries, or other surface characteristics generated from different depth representations may be compared to evaluate how well the depth information captures the geometry of a resection cavity. The depth information determined from the optical image data may then be used in subsequent processing, such as segmentation of a cavity region, generation of a surface mesh, volumetric analysis, or determination of resection-progress information relative to a planned resection region. FIG. 6 is illustrative, and other depth-estimation, depth-completion, or three-dimensional reconstruction techniques may be used.

### Identifying Resection Regions

Some embodiments may use a deep learning method to create segmentation masks of resection regions of brain tissue. Some embodiments may use a vision transformer model, such as SAM2, by Meta with an image encoder, memory attention, masked autoencoder transformer model, prompt encoder, memory encoder, memory bank, and mask decoder. The system, in some embodiments, may accept input from the surgeon via a prompt encoder to improve segmentation accuracy and correct errors. Some embodiments may create a segmentation mask of a brain surface. Some embodiments may segment both right and left images for redundancy and to get a more complete point cloud.

In some embodiments, the computing system may perform video-frame segmentation on two-dimensional image data (like stereoscopic video) captured during a surgical procedure to distinguish tissue associated with a resection cavity from adjacent tissue, fluids, instruments, or other content appearing in the field of view. The image data may include sequential frames of a video stream from a surgical microscope, exoscope, endoscope, or another optical imaging device positioned to observe the anatomical region. In some cases, the computing system may receive individual frames, frame groups, or a continuous stream and may apply a segmentation pipeline to each frame independently or to multiple temporally adjacent frames together. The output may include a pixel-wise label map, one or more binary masks, one or more multi-class masks, contour data, per-pixel confidence values, or combinations thereof. In some implementations, the segmented region may correspond to tissue already removed, tissue bounding the cavity, tissue remaining within a surgeon-planned resection region, or other regions used later in resection-progress analysis. In some embodiments, the resection region may be planned by a machine learning model or artificial intelligence model, and in some cases, such planning may be performed with the segmentation models described herein. The resulting segmentation output may be stored with frame identifiers and timing information and may be passed to subsequent depth reconstruction, three-dimensional surface labeling, mask generation, or volumetric comparison operations.

In some embodiments, the segmentation pipeline may be implemented with one or more trained image models that operate on video frames of the surgical scene. Suitable model families may include convolutional architectures such as U-Net, U-Net++, or nnU-Net, transformer-based architectures such as SegFormer, Mask2Former, or UNETR, instance-segmentation architectures such as Mask R-CNN, and foundation-model architectures such as Segment Anything Model (SAM), SAM2, or SAM3. In some cases, the computing system may use one model to generate an initial segmentation proposal and may use another model to refine boundaries, suppress false positives, or separate adjacent structures. In some implementations, a first model may classify broad tissue categories, and a second model may refine a cavity boundary at higher spatial resolution. In some cases, the segmentation model may output logits or class probabilities for each pixel, and the computing system may convert those outputs into one or more masks by thresholding, argmax selection, confidence filtering, morphological cleanup, contour smoothing, hole filling, connected-component filtering, or other post-processing operations. In some embodiments, the computing system may further reject or downweight segmented regions that do not satisfy temporal consistency, stereo consistency, or geometric plausibility checks relative to neighboring frames or corresponding reconstructions.

In some embodiments, training of the segmentation model may be performed using previously captured surgical video obtained from prior procedures. For example, frames or frame sequences from microscope video may be selected from procedures involving tumor resection, vascular malformation resection, epilepsy-related tissue removal, or other forms of surgery in which removed tissue is to be tracked relative to a plan. In some cases, one or more users may hand-label the selected frames to identify cavity interior, cavity boundary, exposed anatomy, blood, instruments, cottonoids, glare regions, smoke, irrigation, or other scene content. In some implementations, the labels may be revised through multiple review passes, and disagreement between annotators may be resolved by a clinician or another reviewer. The computing system may then use the labeled data to train a segmentation model from initialization, fine-tune a previously trained segmentation model, or adapt a foundation model to the intraoperative domain. In some cases, the training pipeline may include augmentation operations such as cropping, scaling, rotation, color perturbation, blur simulation, noise injection, glare simulation, smoke simulation, or temporal subsampling so that the trained model may better handle variations in lighting, tissue appearance, magnification, and imaging conditions encountered during surgery.

In some embodiments, the segmentation pipeline may process image data from a stereoscopic surgical microscope in which first and second optical channels observe the same surgical scene from different viewpoints. The computing system may apply the same segmentation model architecture to both optical channels, may apply separately trained models to the respective channels, or may process the two channels jointly with a model configured to receive stereo image pairs. In some cases, the computing system may determine a first segmentation mask for a first optical channel and a second segmentation mask for a second optical channel and may use the two masks together with stereo correspondence or disparity information to identify portions of a depth map, point cloud, or surface mesh associated with the resection cavity. In some implementations, the computing system may evaluate agreement between the first and second masks after epipolar alignment, disparity-based reprojection, or three-dimensional back-projection, and may revise one or both masks when disagreement exceeds a threshold. In this way, the segmentation output from both stereo perspectives may be used not only to identify cavity pixels in two-dimensional image space but also to determine which portions of three-dimensional reconstructed geometry correspond to removed tissue. In some cases, data structures other than a mesh may be used, examples including a point cloud, a Gaussian splat, and a neural radiance field.

In some embodiments, the computing system may transfer segmentation labels from two-dimensional image space onto three-dimensional geometry reconstructed from the intraoperative imagery. For example, after determining depth values from stereo imagery or another reconstruction process, the computing system may reproject pixels labeled as cavity or non-cavity along corresponding camera rays into a reconstruction coordinate system. In some cases, the computing system may assign labels to points in a point cloud, to vertices of a mesh, to triangular faces of a mesh, or to voxels in an intermediate three-dimensional occupancy representation. Where a surface mesh includes tens of thousands of polygons or more, the computing system may determine, for each face or vertex, whether a projected image location from one or more segmented frames overlaps that face or vertex and may assign a label and a confidence score accordingly. In some implementations, labels from multiple frames may be aggregated by weighted voting, confidence accumulation, temporal filtering, or visibility-aware fusion so that cavity regions remain stable even when individual frames contain transient occlusions or imperfect boundaries. The resulting labeled three-dimensional representation may then be used for cavity closing operations, resection-mask generation, and comparison against a planned resection region.

In some embodiments, more than two optical viewpoints may be used. For example, the computing system may receive image data from a stereoscopic microscope together with additional cameras, from a microscope repositioned across multiple viewpoints, from a light-field camera, or from another multi-view imaging arrangement in the operating room. The computing system may then perform multi-view reconstruction or structure-from-motion operations to determine a more complete geometric representation of the cavity and surrounding anatomy. In some cases, different viewpoints may expose portions of a cavity that are occluded from a single viewing direction because of cavity depth, overhanging tissue, blood, instruments, or local curvature of the resection surface. The segmentation pipeline may therefore be applied to frames from the additional viewpoints, and the resulting labels may be fused on shared three-dimensional geometry. In some implementations, the computing system may determine which segmented observations correspond to the same physical surface regions and may merge those observations based on view angle, confidence score, reconstruction consistency, or timing. This may allow the cavity representation used in resection-progress analysis to reflect portions of the surgical scene that are not fully observable from one view alone.

In some embodiments, the segmentation and reconstruction pipeline may operate across different microscope hardware configurations and different optical states without requiring that all procedures use the same camera model, zoom setting, working distance, or calibration state. For example, the computing system may receive metadata describing zoom, focus, focal length, pose, or calibration when such metadata is available, but some implementations may additionally or alternatively determine scale relationships, pose relationships, or correspondence relationships from image content, stereo geometry, navigation data, multi-view consistency, or combinations thereof. In some cases, the segmentation model may be trained on image data collected from different microscopes, different magnification settings, and different lighting conditions so that the model remains operable despite hardware variation. In some implementations, normalization operations may be applied to incoming frames before segmentation, such as resizing, histogram normalization, lens-distortion correction, cropping, or color-space conversion. The segmentation output may therefore be used as part of a pipeline that remains operable across different microscope systems and different zoom positions while still producing image-space or geometry-space cavity labels suitable for later registration and progress measurement.

In some embodiments, the segmented cavity information may be used to determine one or more progress-related outputs relative to a preoperative plan, as described below.

### Volumetric Analysis of Resection Regions

To calculate the volume of the resection cavity, its shape may first be defined in some embodiments. The segmentation mask of the resection cavity can be applied to the depth map to leave only the points of the cavity. The perimeter of the segmentation mask forms a closed shape.

The point cloud, in some embodiments, can be turned into a mesh by a meshing algorithm such as alpha shapes, ball pivoting, normal estimation, or Poisson surface reconstruction. The “top” of the cavity, in some embodiments, can be closed by filling with points, e.g., uniformly, prior to creating the mesh. Some embodiments may use the segmentation mask of the brain surface to approximate a curved brain surface to close the cavity volume.

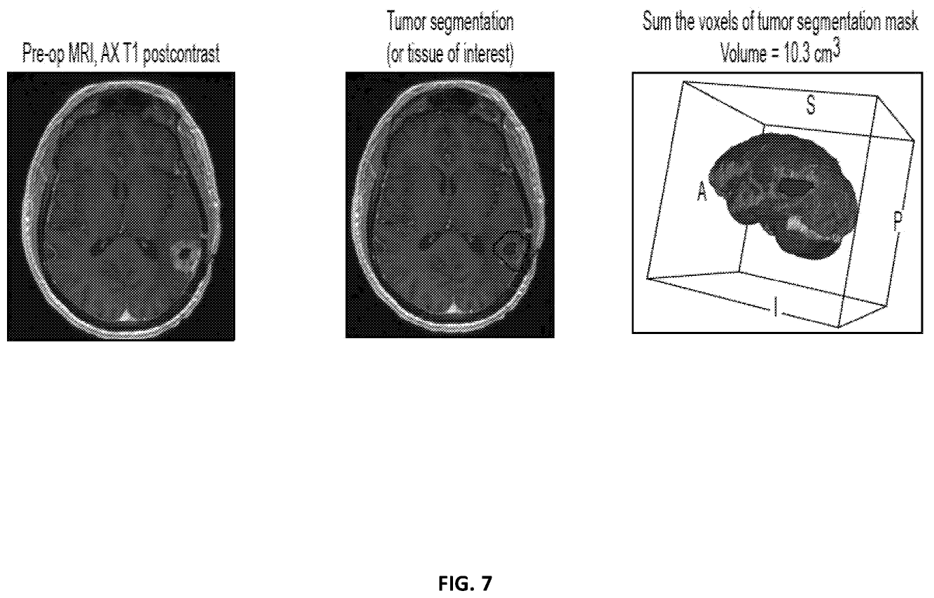

FIG. 7 shows example determination of a target volume or planned resection region from volumetric image data. In some embodiments, the volumetric image data may include preoperative magnetic resonance imaging, computed tomography data, or other volumetric imaging of an anatomical region. The volumetric image data may be processed to determine a lesion region, an organ region, a planned resection region, or combinations thereof. In some embodiments, segmentation of the volumetric image data may be performed with one or more trained models, by manual annotation, or with a combination of automated processing and user input. The resulting data may be used to generate a three-dimensional representation of tissue associated with a planned resection and to form one or more masks for subsequent comparison with intraoperative resection information.

In some embodiments, the planned resection region shown in FIG. 7 may be surgeon-defined or surgeon-edited and need not be coextensive with a lesion boundary. For example, where the tissue being treated includes a tumor, the planned resection region may extend beyond the tumor boundary in one portion of the anatomical region and may remain within the tumor boundary in another portion of the anatomical region. The target volume or planned resection region determined from the volumetric image data may then be used in later processing to assess resection progress, determine tissue remaining within the plan, or determine tissue removed outside the plan.

### Planning the Surgical Target

A target for a surgery may be determined in advance of the surgery based on pre-operative imaging. In some embodiments, a resection plan may be created before surgery by imaging the patient with one or more preoperative imaging modalities and processing the resulting image data to define a target region for tissue removal. The imaging data may include magnetic resonance imaging, computed tomography, positron emission tomography, ultrasound, angiographic imaging, diffusion imaging, perfusion imaging, spectroscopy-derived imaging, fluorescence-related imaging, or combinations thereof. In some cases, the preoperative image data may be represented as a three-dimensional voxel array in which respective voxels correspond to spatial locations within an anatomical region of the patient. The computing system may receive one or more such volumetric datasets, may register different datasets into a shared coordinate system, and may present the data in slice views, rendered views, contour views, or other interfaces through which a user may inspect anatomy and define a resection target. The resulting resection plan may be stored as one or more masks, contours, meshes, label maps, region annotations, or other data structures associated with the preoperative image space.

In some embodiments, the resection plan may be specific to the disease, disorder, lesion, or condition being treated and may therefore correspond to different target definitions for different procedural contexts. For example, the target region may correspond to a malignant tumor, a low-grade tumor, a vascular malformation, an epileptic focus, an abscess, a necrotic region, an injured tissue region, an entire segment of an organ, an entire lobe of an organ, or another anatomical portion selected for removal. In some cases, the target may correspond to tissue identified as pathologic in the imaging data. In some implementations, the target may include both pathologic tissue and additional surrounding tissue selected for removal based on a treatment objective, anticipated spread pattern, anatomical margins, or other planning considerations. In some cases, the target may exclude portions of abnormal tissue that are identified in the imaging data but are not selected for removal because of anatomical constraints, functional constraints, or other surgical considerations reflected in the preoperative plan.

In some embodiments, the computing system may determine one or more segmentations from the preoperative image data as part of creating the resection plan. The segmentation may be directed to a lesion boundary, an organ boundary, one or more adjacent structures, a treatment margin, or combinations thereof. In some cases, the segmentation may be performed with a deep-learning model that accepts the volumetric image data as input and outputs one or more voxel-wise label maps corresponding to lesion tissue, organ tissue, edema, vascular structures, white-matter tracts, functional areas, or other structures represented in the image data. Suitable models may include three-dimensional convolutional networks, encoder-decoder networks, transformer-based volumetric segmentation networks, or hybrid architectures that process image volumes, image patches, or slice groupings. In some implementations, the computing system may use a pretrained model configured to detect one or more lesion types and their approximate boundaries in magnetic resonance imaging or other volumetric imaging data. For example, a pretrained model may process the volumetric image data to identify a high-grade glioma candidate region, a contrast-enhancing tumor region, a non-enhancing infiltrative region, a necrotic core, or surrounding edema, after which one or more of those outputs may be used in generating a candidate resection region.

In some embodiments, manual labeling may be used instead of, or together with, automated segmentation. For example, the computing system may present axial, coronal, sagittal, oblique, curved-planar, or rendered three-dimensional views of the preoperative image data and may receive user input indicating boundaries of tissue to be removed. In some cases, a surgeon or another user may draw contours on selected slices, paint labels onto regions, place seed points for region growing, approve or reject model-generated boundaries, drag boundary points, add or subtract subregions, or otherwise revise a candidate segmentation. In some implementations, the computing system may interpolate between manually labeled slices, smooth a contour sequence, propagate edits through adjacent slices, or update a three-dimensional mask in response to a user edit on a two-dimensional view. In this way, the resection plan may reflect both image-derived tissue characterization and surgeon-selected modifications indicating where cutting is to proceed or where tissue is to remain.

In some embodiments, creation of the resection plan may include both automated lesion identification and surgeon-directed editing of a candidate target region. For example, the computing system may first process the volumetric image data with one or more trained models to determine a candidate lesion mask and may then provide tools through which the surgeon may inspect and revise the candidate mask. In some cases, the user interface may allow the surgeon to view the candidate region relative to contrast enhancement, edema, vascular anatomy, cortical anatomy, eloquent cortex, white-matter pathways, or other structures represented in the imaging data or in additional registered datasets. In some implementations, the surgeon may expand a boundary in one region, contract a boundary in another region, exclude a subregion near a functional boundary, or include additional tissue outside an automatically detected lesion boundary. The computing system may then generate a revised plan mask reflecting those edits and may store the revised plan mask as the operative resection plan used later in intraoperative comparison. In some cases, different voxels may be labeled as part of the planning process to indicate whether they are to be removed, avoided, treated in some manner, or correspond to particular structures.

In some embodiments, when the surgery relates to tumor resection, the resection plan need not match the segmented tumor boundary exactly. For example, the plan may extend beyond the tumor boundary in one portion of the anatomical region so that the target includes additional surrounding tissue selected for supramaximal resection. In some cases, the additional tissue may be selected by applying a nonuniform margin, by editing a candidate boundary outward in selected locations, by following one or more anatomical corridors, or by extending the region along tissue expected to contain infiltrative disease. In some implementations, the computing system may allow the surgeon to specify different outward offsets in different local regions, to draw asymmetric expansion contours, or to approve system-generated expansions based on tumor appearance, anatomical context, or lesion-class-specific planning rules. The resulting plan may therefore include tissue outside the visible or segmented tumor in one or more selected areas of the preoperative image space.

In some embodiments, the resection plan may also be smaller than the segmented tumor in one or more areas. For example, the computing system may allow the surgeon to retract the target boundary inward relative to the tumor boundary where preservation of eloquent brain tissue, vascular structures, white-matter pathways, cranial nerves, or other adjacent structures is desired. In some cases, inward adjustment of the boundary may be performed to reduce surgical risk, to avoid expected postoperative deficit, to preserve organ function, or to reflect access limitations identified during preoperative review. In some implementations, the computing system may present overlaid data from functional magnetic resonance imaging, diffusion tractography, angiography, cortical mapping data, prior annotations, or other registered datasets, and the surgeon may use those additional views to edit the resection boundary so that the plan avoids selected regions. The resulting resection plan may therefore be larger than a tumor boundary in some areas and smaller than the tumor boundary in other areas within the same case.

In some embodiments, once the target region has been defined, the computing system may convert the resection plan into one or more computational representations used later during the procedure. For example, the plan may be stored as a volumetric plan mask on the voxel grid of the preoperative imaging data, as a surface mesh bounding the target region, as a contour set associated with slice locations, or as multiple linked representations that may be transformed between coordinate systems. In some cases, the computing system may also determine related masks or region representations for an organ boundary, lesion boundary, no-go structures, or other anatomical regions that may later be used in comparison with an intraoperatively determined resection mask. The stored resection plan may then serve as the preoperative reference against which subsequently observed tissue removal is measured during the surgical procedure.

Comparing Resection Volumetric Analysis Vs. Target

Once the resection cavity has been closed via meshing algorithm, its volume can be calculated, in some embodiments, by breaking down the mesh into individual triangles, and then calculating the “signed volume” of each triangle relative to an arbitrary reference point. Some embodiments may then sum up the signed volumes of all triangles.

The measured volume may be determined with the following equation: Estimated extent of resection=Resection cavity volume/Tumor volume from MRI segmentation

In some embodiments, volumetric comparison is not limited to a single ratio between a cavity volume and a lesion volume. Rather, the computing system may determine a plan mask corresponding to a planned resection region, a resection mask corresponding to tissue determined to have been removed, and an organ-region mask corresponding to an anatomical region within which the analysis is constrained. Using these masks in a shared spatial frame, the computing system may determine a progress mask corresponding to an intersection of the resection mask and the plan mask, a remnant mask corresponding to a portion of the plan mask outside the resection mask, and a supra-planned mask corresponding to a portion of the resection mask outside the plan mask and within the organ-region mask. The computing system may then determine separate output metrics from these respective masks, such as a progress volume corresponding to tissue removed from within the planned resection region, a remnant volume corresponding to tissue remaining within the planned resection region, a progress fraction based on the relative amounts of within-plan removed and within-plan remaining tissue, and a supra-planned resection volume corresponding to tissue removed outside the planned resection region but within the anatomical region under analysis. In this way, the volumetric comparison may distinguish between within-plan progress, within-plan remaining tissue, and outside-plan removal, rather than representing resection progress only as a single cavity-to-lesion volume relationship.

### Matching Process—Defining 2D Rendered Brain Perspective

Various processes may be used to produce a 2D (or 3D) rendered brain perspective, e.g., for display in a UI. Examples follow.

Some embodiments may operate based on processing spatial masks of anatomic feature segmentation includes the following steps:
a. Define a point within the brain (e.g., centroid, anatomic landmark, or otherwise derived from patient shape)
b. Define a point within the tumor (e.g., centroid or other point derived from its shape)
c. Use these points to define a vector that defines the camera viewing angle

Some embodiments may operate based on neuronavigation and includes calculations using position of microscope camera relative to patient.

### Reprojecting the Segmentation Mask

In some embodiments, after intraoperative image data has been segmented in two-dimensional image space, a computing system may transfer the resulting pixel labels onto a three-dimensional surface mesh generated from the same surgical scene. The image data may include first and second image streams from a stereoscopic surgical microscope, and the three-dimensional surface mesh may be generated from stereo reconstruction of those image streams. In some cases, the segmentation output may indicate, on a pixel-by-pixel basis, whether image regions correspond to a resection cavity, cavity boundary tissue, adjacent anatomy, blood, instruments, glare, or other scene content. The computing system may then use those pixel-level labels to determine which portions of the reconstructed three-dimensional geometry correspond to the resection cavity. In some implementations, the reconstructed geometry may be represented as a polygon mesh having a large number of triangular faces, such as many thousands or tens of thousands of faces, and the computing system may assign one or more labels to individual faces, vertices, or both.

In some embodiments, the computing system may determine a spatial relationship between the segmented image and the three-dimensional surface mesh using camera parameters associated with the intraoperative imaging device. The camera parameters may include intrinsic parameters, extrinsic parameters, stereo rectification parameters, projection matrices, focal parameters, principal-point parameters, distortion parameters, pose information, baseline information between stereo cameras, or combinations thereof. In some cases, the three-dimensional surface mesh may initially be represented in a camera-relative coordinate system derived from stereo reconstruction, and the computing system may therefore project mesh vertices or mesh faces into the image plane of one or more source images from which the mesh was reconstructed. In some implementations, the computing system may evaluate, for a given triangular face, where each vertex of that face maps onto the image plane, may determine a projected polygon corresponding to that face in image coordinates, and may compare that projected polygon with one or more segmented pixel regions. In some cases, the computing system may determine that a face is associated with the resection cavity when at least a threshold portion of the projected polygon overlaps pixels labeled as cavity pixels in the segmentation mask.

In some embodiments, the reprojection may be performed at the vertex level before face labels are assigned. For example, the computing system may project each vertex of the mesh into the image plane, may sample a segmentation label at the projected pixel location or within a neighborhood of that location, and may assign one or more candidate labels and one or more confidence values to the vertex. In some implementations, once candidate labels have been assigned to the vertices of a triangular face, the computing system may determine a face label based on the labels of the vertices. In some cases, a face may be labeled as cavity-associated when all three vertices are labeled as cavity-associated, when a majority of the vertices are labeled as cavity-associated, when an interpolated interior area of the projected face overlaps cavity pixels above a threshold, or when a weighted score derived from the vertex labels exceeds a threshold. In some implementations, the computing system may store both a categorical label and a confidence score for each face so that later volumetric processing may use the confidence score to accept, reject, or downweight portions of the mesh.

In some embodiments, the computing system may perform the reprojection using both stereo views rather than a single view. For example, a first segmentation mask may be generated for a first optical channel of a stereoscopic microscope and a second segmentation mask may be generated for a second optical channel of the stereoscopic microscope. The computing system may then project a mesh face into both image planes and may determine a first candidate label from the first segmentation mask and a second candidate label from the second segmentation mask. In some cases, when the first and second candidate labels agree, the computing system may assign the agreed label to the face with an increased confidence value. In some implementations, when the first and second candidate labels disagree, the computing system may resolve the disagreement based on visibility, reprojection error, segmentation confidence, angle of view, occlusion state, or other criteria. In some cases, the computing system may reject a candidate label from one view when the face is back-facing, occluded, or poorly observed in that view, while retaining the candidate label from the other view. In this manner, the computing system may use both stereo perspectives to determine which mesh regions correspond to removed tissue while reducing the effect of noise, partial occlusion, or view-specific segmentation error.

In some embodiments, the reprojection process may include a visibility analysis before assigning labels to mesh faces. For example, the computing system may determine whether a projected face is actually visible from a camera viewpoint by using depth ordering, z-buffer comparison, ray-intersection testing, or another occlusion-checking process. In some cases, this may reduce assignment of cavity labels to mesh faces that project into a segmented region in image space but are hidden behind another portion of the reconstructed geometry in the corresponding view. In some implementations, the computing system may further exclude image regions associated with instruments, blood, glare, smoke, or other transient obstructions before assigning labels to the mesh. The computing system may therefore assign face labels based on segmented image content that is both geometrically corresponding and visible, rather than based only on two-dimensional overlap in the image plane.

In some embodiments, the computing system may aggregate label information across multiple frames of intraoperative video rather than relying on a single frame. For example, the three-dimensional surface mesh may be reconstructed from multiple stereo pairs captured over a short interval, and the computing system may project mesh faces into a corresponding set of segmented frames. In some cases, each frame may yield a candidate label and confidence value for a given face. The computing system may then combine those candidate labels using weighted voting, temporal smoothing, maximum-confidence selection, probabilistic fusion, or another aggregation process. In some implementations, observations from frames having lower blur, lower motion, lower occlusion, or higher segmentation confidence may be weighted more heavily than observations from frames having lower image quality. In some cases, the computing system may thereby produce a labeled mesh in which individual triangular faces are associated with cavity or non-cavity labels derived from repeated observations of the same physical surface region over time.

In some embodiments, the resulting labeled three-dimensional surface mesh may include a first set of triangular faces corresponding to the resection cavity and a second set of triangular faces corresponding to surrounding anatomy or other non-cavity regions. The computing system may then use the subset of faces labeled as cavity-associated to define a cavity surface for later mesh-repair operations, closure operations, voxelization operations, or comparison with a preoperative resection plan. In some implementations, where the original surface mesh includes mixed or uncertain labeling near a cavity boundary, the computing system may refine the labeled cavity surface by removing isolated mislabeled faces, smoothing label transitions across neighboring faces, enforcing connectivity constraints, or retaining a largest connected cavity-associated component. The labeled mesh may therefore provide an intermediate representation in which segmentation information originally determined in two-dimensional intraoperative image space has been transferred onto three-dimensional reconstructed geometry in a form suitable for later volumetric analysis.

Register 3D reconstruction from in-surgery imaging to pre-operative imaging 3D model

In some embodiments, after the cavity-associated surface mesh has been determined from intraoperative optical image data, a computing system may register that three-dimensional reconstruction to a three-dimensional model derived from preoperative imaging so that the intraoperative mesh and the preoperative plan occupy a shared spatial frame. The intraoperative reconstruction may initially be expressed in a coordinate system tied to the optical imaging device, such as a camera-relative coordinate system in which positions are defined relative to an origin of a stereoscopic microscope, an exoscope, or another intraoperative imaging assembly. In some cases, distances in that coordinate system may be determined only up to a reconstruction scale, may depend on camera calibration state, or may otherwise not yet correspond directly to physical dimensions used in the preoperative imaging dataset. The preoperative model, by contrast, may be represented in scanner coordinates, patient coordinates, or voxel coordinates associated with magnetic resonance imaging, computed tomography, or another volumetric imaging modality obtained before surgery. The computing system may therefore determine one or more transformations that scale, rotate, and translate the intraoperative mesh so that the mesh is aligned with the preoperative three-dimensional model.

In some embodiments, the preoperative three-dimensional model to which the intraoperative mesh is registered may be generated from the preoperative volumetric image data before surgery. For example, the computing system may segment one or more anatomical surfaces, lesion surfaces, cavity-adjacent surfaces, cortical surfaces, organ boundaries, vessel structures, ventricular boundaries, or other features from the preoperative image volume and may convert one or more of those segmentations into a surface mesh, point cloud, signed-distance representation, volumetric label map, or another geometry representation. In some cases, the preoperative model may include only an organ surface or exposed anatomical surface. In some implementations, the preoperative model may additionally include the resection plan model, lesion model, or one or more no-go structures that may later be used in comparison with the resection mask. The computing system may thereby prepare a reference geometry in a coordinate system that is already associated with the preoperative image volume and that may serve as a target for registration of the intraoperative mesh.

In some embodiments, the computing system may determine an initial alignment between the intraoperative mesh and the preoperative model before performing a finer registration. For example, the computing system may receive pose information for the optical imaging device from a navigation system and may receive patient-registration information that relates a patient reference frame to the preoperative image space. In some cases, the navigation system may provide a pose of the microscope or stereoscopic camera assembly relative to a tracked reference fixed to the patient, and the patient reference may already be registered to the preoperative image volume using fiducials, anatomical landmarks, surface matching, or another patient-registration procedure. The computing system may then compose a chain of transformations from the camera-relative reconstruction frame to the patient frame and from the patient frame to the scanner frame of the preoperative image data. In some implementations, this composed transformation may provide an initial rotation and translation for placement of the intraoperative mesh near the corresponding anatomy in the preoperative model before later refinement operations are performed.

In some embodiments, the computing system may determine scale for the intraoperative mesh as part of the registration process. Where the stereo reconstruction has already been generated using a known baseline and known intrinsic parameters, the resulting mesh may already approximate physical dimensions, and the scale component of the transformation may be near unity. In some cases, however, residual scale error may remain because of zoom changes, incomplete calibration, monocular reconstruction, structure-from-motion reconstruction, or another reconstruction path in which scale is not fixed directly in millimeters. The computing system may then determine a scale factor by comparing one or more distances measured on the intraoperative mesh with corresponding distances in the preoperative model. For example, the computing system may identify pairs of anatomical landmarks, surface extents, vessel branch points, sulcal intersections, cavity-adjacent ridges, fiducial markers, or other corresponding features and may determine a scale that reduces discrepancy between inter-feature distances. In some implementations, the computing system may estimate scale jointly with rotation and translation by optimizing a similarity transformation between the intraoperative mesh and the preoperative model. In some cases, the similarity transformation may be initialized from navigation information and refined by repeated correspondence and update operations until residual alignment error is reduced below a threshold or until another stopping condition is satisfied.

In some embodiments, the computing system may determine correspondences between the intraoperative mesh and the preoperative model based on surface geometry. For example, the computing system may sample points from the intraoperative mesh, may determine surface normals for those sampled points, and may identify candidate corresponding points or surface patches on the preoperative model. In some cases, the candidate correspondences may be determined using nearest-neighbor searches in three-dimensional space, nearest-neighbor searches constrained by surface-normal similarity, feature-descriptor matching, curvature matching, geodesic-pattern matching, or combinations thereof. In some implementations, the computing system may reject candidate correspondences that exceed a distance threshold, that have inconsistent normal directions, that lie in masked or excluded regions, or that otherwise appear unlikely to represent the same anatomical structure. The computing system may then solve for an updated transformation that reduces spatial discrepancy between the intraoperative samples and the corresponding locations on the preoperative model. In some cases, the discrepancy measure may be based on point-to-point distance, point-to-plane distance, surface-to-surface distance, or another geometric error metric. The computing system may repeat the correspondence determination and transformation update iteratively so that the intraoperative mesh progressively approaches the preoperative model in position, orientation, and scale.

In some embodiments, a closest-point registration process may be used. For example, the computing system may begin with an initial estimate of a similarity transformation, may transform the intraoperative mesh according to that estimate, may determine for each of a set of transformed sample points a nearest point on the preoperative surface, and may solve for an updated scale, rotation, and translation that reduce aggregate distance between the transformed sample points and the corresponding preoperative points. In some cases, the aggregate distance may be weighted so that points with higher reconstruction confidence, lower segmentation uncertainty, or better local visibility contribute more heavily than points having lower confidence. In some implementations, points near likely artifacts, such as regions influenced by instruments, glare, pooled blood, or unstable stereo depth, may be downweighted or excluded. The computing system may repeat these operations across multiple iterations and may stop when the change in transformation falls below a threshold, when average residual error ceases to improve, or when a maximum iteration count is reached. In some cases, the resulting transformation may be treated as a rigid-plus-scale alignment that places the intraoperative mesh into scanner coordinates of the preoperative image dataset.

In some embodiments, feature-based registration may be used instead of, or together with, closest-point surface registration. For example, the computing system may identify salient anatomical features on the intraoperative mesh, such as ridge lines, sulcal boundaries, vessel grooves, exposed cortical landmarks, boundary curves of exposed anatomy, or tracked fiducial points, and may identify corresponding features in the preoperative model derived from magnetic resonance imaging or another preoperative imaging modality. In some cases, the computing system may determine descriptors for local surface patches around the candidate features and may match those descriptors across the intraoperative and preoperative representations. In some implementations, the computing system may solve for a transformation that aligns the matched feature pairs while rejecting outliers using a robust-estimation procedure. The robust-estimation procedure may evaluate subsets of candidate matches, may determine candidate transformations from those subsets, may score the candidate transformations based on how many feature pairs remain consistent within a tolerance, and may retain a transformation associated with a larger consensus set. The computing system may then refine that transformation using a denser surface-registration process or a local geometric optimization.

In some embodiments, image-based registration may also be used. For example, the computing system may render the preoperative three-dimensional model from a candidate camera pose and may compare the rendered contours, visible surfaces, or projected anatomical features with contours or segmented regions observed in the intraoperative optical image data. In some cases, the computing system may adjust pose and scale parameters so that projected boundaries from the preoperative model better align with segmented cavity-adjacent tissue boundaries, cortical boundaries, vessel silhouettes, or other features visible in the intraoperative images. In some implementations, the objective function for this registration may penalize contour misalignment, silhouette mismatch, reprojection error of landmarks, mismatch between rendered depth ordering and observed depth ordering, or combinations thereof. The computing system may thereby use information still present in image space, rather than only the reconstructed mesh, to refine the alignment between the intraoperative reconstruction and the preoperative model.

In some embodiments, the registration process may use anatomical regions outside the cavity itself. For example, the cavity surface may not directly correspond to any preexisting surface in the preoperative image because tissue has been removed during the procedure. The computing system may therefore rely on surrounding exposed anatomy, cortical surface regions, organ boundary regions, visible vessels, bone edges, dural edges, or other nonresected structures that are represented both in the intraoperative reconstruction and in the preoperative model. In some cases, the computing system may segment or otherwise identify those nonresected structures in the intraoperative image data, may transfer the corresponding labels to the reconstructed mesh, and may use the labeled surrounding surface regions as anchors for registration. In some implementations, once the surrounding anatomy has been aligned to the preoperative model, the cavity-associated portion of the intraoperative mesh may inherit that same transformation and thereby be placed into the scanner coordinate system together with the surrounding surface.

In some embodiments, the computing system may determine the registration transformation in stages. For example, a first stage may determine a coarse scale and translation from tracked microscope pose and patient registration data. A second stage may refine rotation and translation based on landmark correspondences or surface correspondences. A third stage may refine scale jointly with pose using a similarity-registration process. A fourth stage may apply local deformation to account for intraoperative tissue displacement once the intraoperative mesh has been brought into approximate alignment with the preoperative model. In some cases, the local deformation may be based on a biomechanical model, a finite-element model, a control-point displacement model, a spline-based warping model, or another deformation process that propagates observed surface displacement into deeper tissue regions. In some implementations, the rigid or similarity alignment may therefore serve as a precursor to later deformation of the preoperative anatomical model, rather than as the only alignment operation used in the analysis.

In some embodiments, after the transformation has been determined, the computing system may apply the transformation to vertices of the intraoperative mesh so that the mesh is expressed in scanner coordinates associated with the preoperative image data. In some cases, each vertex of the cavity mesh may be multiplied by a homogeneous transformation that includes scale, rotation, and translation. In some implementations, when the transformation includes nonrigid components, the computing system may evaluate the deformation field, mesh-warping function, or control-point interpolation at each vertex location to determine the transformed position. Once the transformed mesh is represented in scanner coordinates, the computing system may convert those coordinates into voxel coordinates of the preoperative image volume using image-origin information, voxel spacing information, direction cosines, affine transforms, or other metadata associated with the preoperative imaging dataset. The transformed mesh may then be used for later watertight closure, voxelization, resection-mask construction, comparison against a plan mask, or updating of a preoperative anatomical model.

In some embodiments, the computing system may maintain one or more measures of registration confidence or residual error. For example, the computing system may determine an average point-to-surface distance after alignment, a distribution of residual distances for accepted correspondences, a reprojection error for landmark matches, a consistency value across multiple camera views, or another indicator of registration quality. In some cases, the computing system may use those values to determine whether the registration is accepted as-is, whether additional refinement is to be performed, whether a different initialization is to be attempted, or whether one or more later volumetric outputs are to be flagged as lower confidence. In some implementations, the computing system may repeat the registration when new intraoperative viewpoints become available, when additional exposed anatomy is observed, or when subsequent stages of tissue removal materially change the visible surgical scene. The registration may therefore be updated during the procedure so that the cavity mesh from the prior step remains scaled and aligned relative to the preoperative three-dimensional model used for resection-progress analysis.

Deformation of pre-operative model based on surgical progress

In some embodiments, before or after registering an intraoperative surface reconstruction to the coordinate system of preoperative imaging, a computing system may deform a three-dimensional preoperative anatomical model to better represent the current intraoperative state before comparing the observed resection cavity to the planned resection. The computing system may determine a deformed version of the preoperative model by applying a physics-based deformation model constrained by what the computer-vision pipeline observes during surgery and by what the system determines has already been cut or removed.

In some embodiments, the preoperative anatomical model may first be converted into a computational mechanics representation suitable for deformation analysis. For example, the computing system may generate a volumetric mesh from the preoperative image data, such as a tetrahedral mesh, hexahedral mesh, voxel-based finite-difference grid, particle representation, mass-spring representation, or another discretized model of tissue volume. In some cases, the mesh may include nodes at anatomical boundaries, within tissue compartments, and around structures that are to be tracked separately during deformation. In some implementations, different parts of the model may be assigned different mechanical parameters based on tissue class, such as parameters for tumor tissue, normal parenchyma, edema region, scar tissue, necrotic tissue, cerebrospinal-fluid-adjacent regions, vessel-adjacent regions, or organ capsule regions. The computing system may thereby prepare a deformable preoperative model in which node positions, element connectivity, and material properties may be updated as new intraoperative observations become available.

In some embodiments, the computing system may derive boundary conditions for the deformation model from the computer-vision side of the intraoperative pipeline. For example, stereo reconstruction and segmentation of intraoperative microscope imagery may provide a current cavity surface, exposed tissue surface, visible sulcal patterns, exposed vessel locations, cortical boundaries, organ-boundary regions, or other observed anatomical features. After those observed features are registered into the coordinate system of the preoperative image data, the computing system may determine where corresponding surfaces or landmarks are located in the preoperative model and may calculate displacements between the preoperative positions and the current observed positions. In some cases, those displacements may be assigned as positional constraints, soft constraints, target positions, surface contact conditions, or force-equivalent conditions on nodes or elements of the deformable model. In some implementations, the computing system may apply stronger constraints to portions of anatomy directly observed by the camera and weaker constraints to deeper or occluded portions not directly observed.

In some embodiments, the computing system may also update the deformation model based on what tissue has been cut, removed, aspirated, coagulated, or otherwise altered during the procedure. For example, once the intraoperative cavity has been segmented and converted into a resection mask or cavity surface, the system may determine which portions of the preoperative anatomical model correspond to tissue no longer present. In some cases, elements of the volumetric mesh that fall within the resection mask may be removed from the mechanics model, may be marked as void regions, may be assigned substantially reduced stiffness, or may otherwise be excluded from later load-bearing behavior. In some implementations, nodes lying on opposite sides of a newly formed cut surface may have their mechanical coupling reduced or eliminated so that the remaining tissue is permitted to relax into the void created by resection. In some cases, the shape of the cut surface observed from intraoperative computer vision may be imposed directly on the deformable model so that the simulated anatomy includes a cavity boundary consistent with what has actually been removed.

In some embodiments, the deformation model may further account for intraoperative influences other than cutting. For example, the computing system may apply loading or relaxation conditions associated with gravity, tissue retraction, cerebrospinal-fluid loss, swelling, pressure release, instrument contact, patient head orientation, or local support from surrounding anatomy. In some cases, gravity direction may be determined from patient pose or operating-room setup, and the system may solve for tissue displacement under that loading after elements corresponding to removed tissue have been eliminated or relaxed. In some implementations, regions attached to relatively fixed anatomy, such as bone-adjacent regions or less-mobile attachment zones, may be constrained to move less than more mobile exposed tissue. In some cases, the computing system may incorporate one or more measured or estimated retractor positions or tool-contact regions into the deformation model so that local tissue displacement caused by surgical manipulation may be reflected in the updated preoperative model.

In some embodiments, the computing system may solve the deformation problem using a finite-element method, a mass-spring solver, a position-based dynamics solver, a reduced-order deformation model, a spline-based warping model with mechanical regularization, or another physics-informed update process. For example, where a finite-element method is used, the computing system may assemble system equations for the remaining tissue elements after resection-related element removal or stiffness reduction, may apply boundary constraints derived from the observed cavity and exposed surface, and may solve for nodal displacements throughout the modeled anatomy. In some cases, the solver may operate linearly for smaller deformations or may include nonlinear updating for larger deformations, contact-like boundary conditions, or material-property changes. In some implementations, the system may solve on a graphics processing unit in the operating room or on another accelerated computing platform so that an updated model may be produced quickly enough to support repeated intraoperative updates.

In some embodiments, the deformation need not be driven solely by direct surface matching. For example, the computing system may use the observed cavity surface from stereo reconstruction as one set of constraints and may use inferred interior deformation as another component computed by the physics model. In some cases, only a portion of the organ surface or cavity boundary is directly observed by the microscope, while deeper tissue and tissue outside the field of view are not visible. The computing system may therefore use the physics model to propagate observed displacement from visible surface regions into unobserved internal regions of the preoperative model. In some implementations, this may allow the lesion boundary, planned resection boundary, ventricular surface, tract locations, or other subsurface structures to be repositioned in a manner consistent with the observed cavity geometry and the current cut state even though those structures are not directly visible in the surgical video.

In some embodiments, once the deformation solution has been determined, the computing system may warp one or more preoperative structures into a current intraoperative configuration. For example, node displacements of the deformable model may be applied to a tumor mesh, a lesion segmentation, a plan mask, an organ-region mask, a vessel model, or a functional-region model derived from the preoperative image data. In some cases, the computing system may resample the preoperative volumetric dataset through the displacement field so that the image volume itself is deformed into an updated anatomical model. In some implementations, the plan mask associated with the intended resection may be warped by the same deformation field so that comparison against the intraoperative cavity occurs relative to a shifted and deformed target rather than relative to the undeformed preoperative anatomy.

Closing the cavity volume of the surface mesh created from in-surgery images

In some embodiments, after a cavity-associated surface has been determined from intraoperative image data, a computing system may process that surface so that the surface bounds a closed three-dimensional region suitable for volumetric analysis. As noted above, the cavity-associated surface may initially be represented as a polygon mesh, such as a triangular mesh derived from stereo reconstruction and labeling of cavity-related regions in the intraoperative images. In some cases, that mesh may not be watertight. For example, the mesh may include one or more open boundaries caused by incomplete depth recovery, segmentation irregularities, occlusion by instruments, local glare, blood, steep cavity geometry, or limited visibility from one or more viewpoints. The computing system may therefore perform mesh-processing operations that identify open boundaries, distinguish a larger opening associated with the exposed mouth of the resection cavity from one or more smaller defects, and then close those openings so that the mesh encloses a volumetric region.

In some embodiments, the computing system may identify open boundaries of the mesh by determining boundary edges and chaining connected boundary edges into closed or partially closed boundary loops. A boundary edge may correspond to an edge incident to fewer than two faces of the current mesh representation. The computing system may traverse adjacency relationships among such boundary edges to determine one or more loops, with each loop represented by an ordered sequence of vertices, edges, or both. In some cases, the mesh may include multiple loops. For example, a first loop may correspond to the upper opening of the cavity where the cavity remains open toward the camera viewpoint, while one or more additional loops may correspond to smaller holes, tears, discontinuities, or local defects elsewhere in the mesh. In some implementations, the computing system may determine one or more measures for each loop, such as total edge-path length, perimeter, enclosed area, bounding-box extent, average normal direction, centroid location, or combinations thereof, and may store those measures for later ranking and classification of the loops.

In some embodiments, the computing system may rank the identified boundary loops according to length or another size-related measure to distinguish a candidate cavity opening from smaller mesh defects. For example, the loop having the greatest total edge-path length may be treated as corresponding to the main opening of the cavity, while one or more loops having smaller lengths may be treated as local holes or tears. In some cases, ranking may additionally account for enclosed area, spatial position relative to the camera direction, orientation relative to a local surface normal, or adjacency to faces labeled as cavity-associated. In some implementations, the computing system may determine that a loop is a defect loop when the loop is below a threshold perimeter, encloses a relatively small area, lies away from an expected cavity mouth region, or satisfies another defect-classification criterion. In this manner, the computing system may process smaller discontinuities differently from the larger opening associated with the cavity mouth.

In some embodiments, the computing system may close one or more smaller loops by constructing a local patch across each such loop. For example, a hole-closure operation may generate a fan mesh spanning the vertices of a given loop. In some cases, the computing system may determine a center point for the fan mesh based on the vertices of the loop, such as by calculating an average of the coordinates of the loop vertices, a centroid of the loop vertices, a weighted centroid, or another representative interior point. The computing system may then generate triangular faces between the center point and successive pairs of adjacent vertices along the loop so that the loop is spanned by a set of triangles. In some implementations, the center point may first be projected onto a local best-fit plane for the loop or adjusted along a local normal direction before the triangular faces are generated. In some cases, the computing system may evaluate the resulting patch for self-intersection, inverted normals, or excessive aspect ratio and may revise the center point or the triangulation accordingly. As a result, smaller defects in the mesh may be closed so that those defects do not remain as openings in the later watertight representation.

In some embodiments, the computing system may process the largest loop differently from smaller loops because the largest loop may correspond to the open mouth of the resection cavity rather than a local defect. For example, the computing system may generate a cap structure based on the largest loop. In some cases, the largest loop may be extruded to form a cap rim. The extrusion may be performed by generating a second loop displaced from the original loop along an extrusion direction. The extrusion direction may correspond to a camera-axis direction, a depth direction, a viewing direction, a local average normal direction, or another selected direction. In some implementations, extrusion along a camera-axis direction may reduce the effect of irregularities at the original loop caused by segmentation error, local dips at the cavity boundary, or portions of an instrument or other obstruction extending into the opening region. The amount of extrusion may be fixed, adaptive, based on loop size, based on local geometry, or determined from another criterion.

In some embodiments, after the cap rim has been formed, the computing system may close the cap rim itself. For example, the cap rim may be closed using a fan mesh in a manner similar to that described for smaller defect loops. In some cases, a center point for the cap rim may be calculated from an average of the coordinates of vertices in the rim loop, from a centroid of the rim loop, or from another representative point associated with the rim. The computing system may then create triangular faces between that center point and adjacent vertex pairs of the rim loop so that the rim is closed by a set of triangles. In some implementations, the system may first regularize the rim loop by smoothing vertex positions, removing small irregular excursions, resampling the loop, or fitting a simplified curve before generating the closing faces. This may allow the cap closure to be less sensitive to local jaggedness present in the original largest boundary loop.

In some embodiments, after the original largest boundary loop and the cap rim have both been defined, the computing system may fill the space between those two loops with triangular faces so that a sidewall of the cap is formed. For example, where the original loop and the extruded rim have corresponding vertex orderings, the computing system may connect successive pairs of vertices from the original loop and the rim loop to form quadrilateral strips, and may divide each strip into two triangular faces. In some cases, where the original loop and the rim loop do not have matching vertex counts, the computing system may resample one or both loops, determine correspondences along arc length, or perform another bridging operation so that the space between the loops may still be triangulated. In some implementations, the resulting bridging faces, together with the original cavity-associated mesh and the cap closure, may form a closed surface that bounds a volumetric interior. In some cases, the computing system may subsequently apply smoothing, remeshing, normal reorientation, manifold enforcement, component filtering, or self-intersection correction so that the resulting mesh is better suited for later voxelization.

In some embodiments, the watertightness of the resulting mesh may be evaluated before the mesh is used for volumetric conversion. For example, the computing system may determine whether each edge is incident to two faces, whether boundary edges remain, whether disconnected components are present, whether face normals are consistently oriented, whether non-manifold vertices or edges remain, or whether self-intersections are detected. In some cases, if the resulting mesh fails one or more watertightness checks, the computing system may perform one or more additional repair operations, such as closing newly identified small loops, merging nearly coincident vertices, retriangulating local patches, removing isolated components, or revising one or more cap faces. In some implementations, the computing system may retain a largest connected component corresponding to the cavity volume and discard small disconnected fragments. The mesh may thereby be converted into a watertight cavity representation that encloses an interior region.

Construct the resection mask from the closed cavity volume

In some embodiments, after a watertight cavity mesh has been determined in scanner coordinates or another physical coordinate system associated with the preoperative volumetric image data, a computing system may construct a resection mask on the voxel grid of that volumetric image. The watertight mesh may represent a closed boundary of tissue determined to have been removed during the procedure, while the volumetric image may be represented as a three-dimensional array of voxels indexed along one or more image axes. To place the watertight mesh into that voxel array, the computing system may convert positions of vertices of the mesh from scanner-space coordinates into voxel-space coordinates. In some cases, the volumetric image data may be associated with an affine mapping that maps voxel indices into physical coordinates, such as millimeter coordinates of a magnetic resonance imaging dataset or another preoperative image dataset. The computing system may apply an inverse of that affine mapping to the vertex coordinates of the watertight mesh so that the mesh is expressed in the coordinate frame of the voxel grid rather than in the physical coordinate frame of the scanner.

In some embodiments, the affine mapping may encode one or more of voxel spacing, image origin, axis orientation, direction cosines, shear, or another linear or translational relationship between image-grid coordinates and physical-space coordinates. The inverse affine transformation may therefore include a matrix component and a translation component that together map a point expressed in scanner coordinates into a point expressed in voxel coordinates. In some cases, the computing system may represent a vertex of the mesh in homogeneous coordinates, may multiply that vertex by the inverse affine transform, and may thereby obtain a corresponding location in continuous voxel space. In some implementations, the transformed coordinates may remain fractional rather than being rounded immediately to integer voxel indices, because later voxelization may evaluate intersections between mesh faces and voxel cells using continuous positions of the transformed vertices. In some cases, the computing system may apply the inverse affine transformation to all vertices of the watertight mesh and may thereby obtain a voxel-space mesh that is aligned to the same image grid later used for plan masks, organ-region masks, lesion masks, or other volumetric representations.

In some embodiments, after the mesh has been expressed in voxel coordinates, the computing system may transform the watertight mesh into a voxel shell corresponding to voxels intersected by the closed surface. For example, the computing system may evaluate triangular faces of the voxel-space mesh and may determine which voxels of the image grid are intersected by those faces or lie sufficiently close to those faces to be treated as part of the mesh boundary. In some cases, the resulting voxel shell may be represented as a three-dimensional array in which voxels corresponding to the surface are assigned an occupied value and other voxels remain unassigned or assigned a background value. In some implementations, the voxelization may be performed by a mesh-processing routine configured to convert a closed triangular mesh into an occupancy shell on a three-dimensional grid, such as a routine analogous to a voxelized function. The shell may therefore represent the boundary of the removed-tissue volume on the same discrete grid used by the preoperative volumetric image data.

In some embodiments, construction of the voxel shell may include one or more resolution-related operations. For example, the computing system may determine shell occupancy at the native resolution of the preoperative image grid, at a resampled resolution, or at another resolution selected for later Boolean operations or volumetric calculations. In some cases, when voxel spacing differs across axes, the shell generation may account for anisotropic voxel dimensions so that occupancy is determined relative to the actual physical extent of the voxels rather than as though all voxels were cubes of equal size. In some implementations, the computing system may clip portions of the transformed mesh that extend beyond bounds of the volumetric image, may discard disconnected fragments that do not belong to a selected cavity component, or may apply one or more cleanup operations to the shell, such as closing local one-voxel discontinuities, removing isolated shell voxels, or regularizing very thin protrusions introduced during voxelization. These or other operations may allow later filling of the shell to proceed on a cleaner discrete boundary representation.

In some embodiments, once the voxel shell has been formed, the computing system may fill the interior of that shell to create a resection mask corresponding to the volume enclosed by the watertight cavity mesh. For example, the computing system may identify voxels representing the exterior of the shell and may propagate an exterior label through connected background voxels, such that background voxels not reached by that exterior propagation may be treated as lying inside the closed shell. In some cases, the filling may be performed by a flood-fill operation, a binary-hole-filling operation, a parity-based interior test, a winding-based fill process, or another interior-determination technique. In some implementations, the filling may be performed by a software routine analogous to a binary_fill_holes function that treats the shell voxels as a closed boundary and assigns interior occupancy to enclosed locations. The filled interior together with, in some cases, the shell voxels themselves may define the volumetric region corresponding to tissue determined to have been removed.

In some embodiments, the resulting resection mask may be stored as a binary volumetric array in which voxels included within the removed-tissue region are assigned logical true values and voxels outside that region are assigned logical false values. In some cases, the mask may instead be stored as an integer array, floating-point array, label map, or another volumetric data structure in which voxels belonging to the resection mask are assigned nonzero intensities and background voxels are assigned zero. In some implementations, the computing system may further associate confidence values, timestamps, or provenance data with the mask, such as identifiers of the intraoperative image sequence from which the mask was derived or quality measures associated with the reconstruction and voxelization stages. The resection mask may then be used in later Boolean operations with a plan mask, an organ-region mask, a lesion mask, or a deformed preoperative anatomical model to determine progress-related volumetric outputs during the surgical procedure.

In some embodiments, after the resection mask has been created, the computing system may perform one or more additional checks before the mask is used in later comparison operations. For example, the computing system may determine whether the mask occupies an implausibly large or implausibly small number of voxels, whether the mask includes disconnected internal components, whether portions of the mask extend outside an expected anatomical region, or whether the mask volume changes abruptly relative to a previous intraoperative measurement. In some cases, the computing system may apply one or more corrective operations, such as retaining a largest connected component, clipping the resection mask to an organ-region mask, smoothing the mask boundary, or regenerating the mask from an earlier shell if a validity criterion is not satisfied. The resulting volumetric resection mask may thereby provide a discrete three-dimensional representation of removed tissue suitable for subsequent volumetric analysis on the same image grid as the preoperative imaging data.

Comparison of resection mask to surgical plan

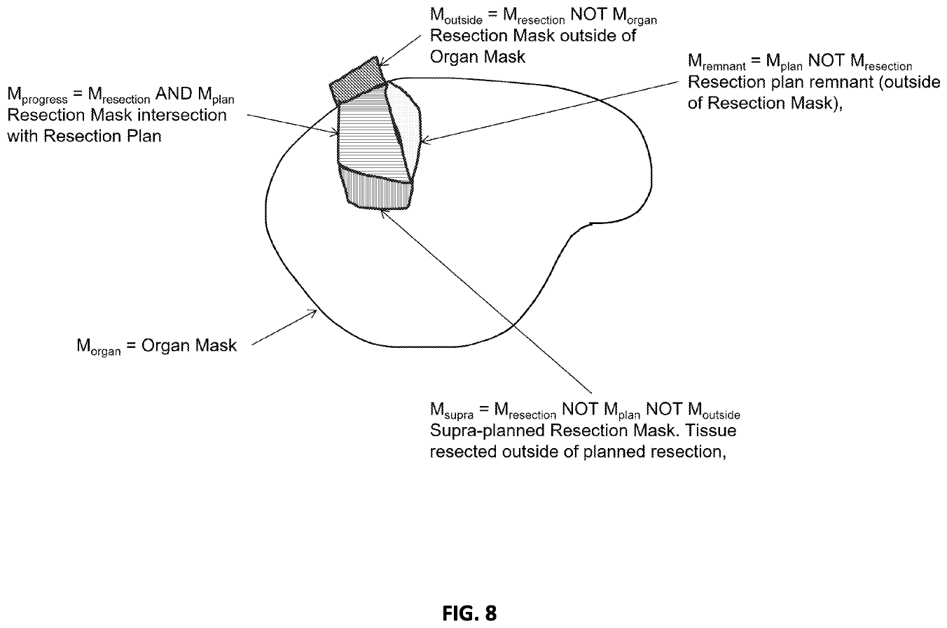

FIG. 8 show examples of ways the voxel-space resection mask formed with the preceding operations may be compared to a surgical plan, like a resection plan, in the same voxel-space. In some embodiments, volumetric progress determination may be based on a resection mask derived from intraoperative image data together with one or more masks derived from preoperative image data, while accounting for the fact that the resection mask may not match the actual removed-tissue volume exactly. For example, the resection mask may be generated from a cavity surface reconstructed from optical image data, followed by mesh closing, voxelization, and interior filling operations. In some cases, those operations may cause the resection mask to include portions that extend beyond a physical cavity boundary, may omit small portions of the cavity, or may include voxels outside the organ or anatomical region intended for analysis. In some implementations, those discrepancies may arise from segmentation irregularities, partial visibility of the cavity, cap-generation operations used to close the mesh, voxel-grid discretization, hole-filling operations, deformation or registration error, or combinations thereof. As a result, some embodiments may avoid treating the total voxel count of the resection mask itself as the sole measure of progress and may instead determine one or more derived masks that constrain the analysis to tissue within the planned resection region or within the organ region being evaluated.

In some embodiments, the computing system may determine a plan mask corresponding to a planned resection region identified from preoperative image data and may determine a resection mask corresponding to tissue inferred to have been removed from intraoperative image data. In some cases, the plan mask and the resection mask may both be represented on a common voxel grid associated with magnetic resonance imaging, computed tomography, or another preoperative volumetric dataset. In some implementations, the computing system may additionally determine an organ-region mask corresponding to the organ, lobe, hemisphere, tissue compartment, or other anatomical region within which the resection-progress analysis is to be constrained. The organ-region mask may be useful where portions of the resection mask extend outside the tissue volume of interest because of reconstruction artifacts, because of the manner in which a cavity cap was formed, or because of discretization effects introduced during voxelization. The computing system may therefore perform Boolean operations on these masks to determine derived volumetric regions that more closely correspond to the progress quantities to be reported.

In some embodiments, the computing system may determine a progress mask corresponding to a portion of the resection mask that overlaps the plan mask. For example, the progress mask may be generated by a Boolean intersection between the resection mask and the plan mask, such that voxels are retained in the progress mask when those voxels are included in both the removed-tissue representation and the planned resection region. In some cases, the resulting progress mask may represent tissue that has been removed from within the intended target region rather than tissue removed anywhere in the vicinity of the cavity. In some implementations, the computing system may then determine a progress volume by counting voxels assigned to the progress mask and multiplying that voxel count by the volume represented by each voxel of the shared image grid. The computing system may likewise determine a plan volume by counting voxels assigned to the plan mask and multiplying that voxel count by the same voxel volume. Progress with respect to the plan may then be expressed as a value derived from the progress volume relative to the plan volume, such as a fraction or percentage indicating how much of the planned region has been removed.

In some embodiments, the computing system may determine progress using a remnant-based approach in addition to, or instead of, the overlap-based approach. For example, the computing system may determine a remnant mask corresponding to the portion of the plan mask that remains outside the resection mask. In some cases, the remnant mask may be generated by retaining voxels that belong to the plan mask while excluding voxels that also belong to the resection mask. The remnant mask may therefore represent tissue that remains within the planned resection region according to the current comparison state. In some implementations, the computing system may determine a remnant volume by counting voxels assigned to the remnant mask and multiplying that voxel count by the physical volume represented by each voxel. The plan volume may be determined in the same manner from the plan mask. Progress may then be expressed as a value derived from the planned volume together with the remnant volume, such as a value indicating how much of the planned region is no longer represented by the remnant mask. In some cases, the remnant-based measure and the overlap-based measure may be mathematically related and may be expected to converge to similar values when the masks are well aligned.

In some embodiments, the computing system may calculate both an overlap-based progress quantity and a remnant-based progress quantity and may present one of them, both of them, or a selected one based on a quality condition. For example, where the resection mask is believed to include some extraneous volume beyond the organ boundary, the overlap-based quantity may more directly represent removed tissue within the planned region because it excludes portions of the resection mask lying outside the plan. In some cases, the remnant-based quantity may be useful because it characterizes what remains of the plan rather than what has been removed. In some implementations, the computing system may compare the two determinations and may use the degree of agreement between them as one indicator of computational consistency. The computing system may also store the underlying masks and voxel counts so that one or more later updates during the same procedure may be compared with earlier updates.

In some embodiments, the computing system may determine a separate quantity corresponding to resection within the organ of interest but outside the planned resection region. This region may be referred to as a supraplanned resection region. In some cases, tissue in that region may have been removed intentionally, such as where the surgeon elects during the procedure to extend the resection beyond the original plan for diagnostic, therapeutic, or access-related reasons. In some implementations, tissue in that region may have been removed unintentionally because the resection deviated from the plan, because the plan was no longer optimal after tissue shift, or because the image-derived cavity boundary includes local extensions beyond the originally planned boundary. The computing system may therefore determine a supraplanned mask so that removal outside the original plan may be quantified separately from within-plan progress.

In some embodiments, determination of the supraplanned mask may include determining an outside-of-organ mask before determining the supraplanned region itself. For example, the outside-of-organ mask may be generated by retaining portions of the resection mask that lie outside the organ-region mask. In some cases, the outside-of-organ mask may identify voxels that should not be counted as removal within the anatomical structure being analyzed, such as voxels introduced outside the organ by cap formation, shell filling, discretization, or registration effects. In some implementations, the computing system may then determine the supraplanned mask by retaining voxels that belong to the resection mask while excluding voxels that belong to the plan mask and also excluding voxels that belong to the outside-of-organ mask. The resulting supraplanned mask may therefore correspond to tissue removed within the organ region but outside the planned resection region. In some cases, this may distinguish removal extending beyond the plan inside the organ from artifacts that fall outside the organ entirely.

In some embodiments, the computing system may determine a supraplanned volume by counting voxels assigned to the supraplanned mask and multiplying that count by the physical volume represented by each voxel. The supraplanned volume may be presented as an independent quantity, may be used together with the progress volume and the remnant volume in a user interface, or may be used to derive additional values such as a ratio between within-plan removal and outside-plan removal. In some cases, the computing system may present a first value corresponding to removed tissue within the plan, a second value corresponding to tissue remaining within the plan, and a third value corresponding to removed tissue within the organ but outside the plan. In some implementations, these values may be updated at multiple time points during the same procedure as new intraoperative image data is acquired and new resection masks are determined.

In some embodiments, Boolean operations used to construct the progress mask, the remnant mask, the outside-of-organ mask, and the supraplanned mask may be performed directly on binary voxel arrays stored in memory. In some cases, voxels having nonzero or logical true values in the respective source masks may be combined by elementwise logical operations across the shared grid. In some implementations, the computing system may first resample one or more masks to a common grid, may clip one or more masks to image bounds, may enforce consistency of axis orientation and voxel spacing, or may otherwise normalize the masks before the Boolean operations are applied. The resulting derived masks may then be stored for later display, further deformation updates, or additional comparison operations during surgery.

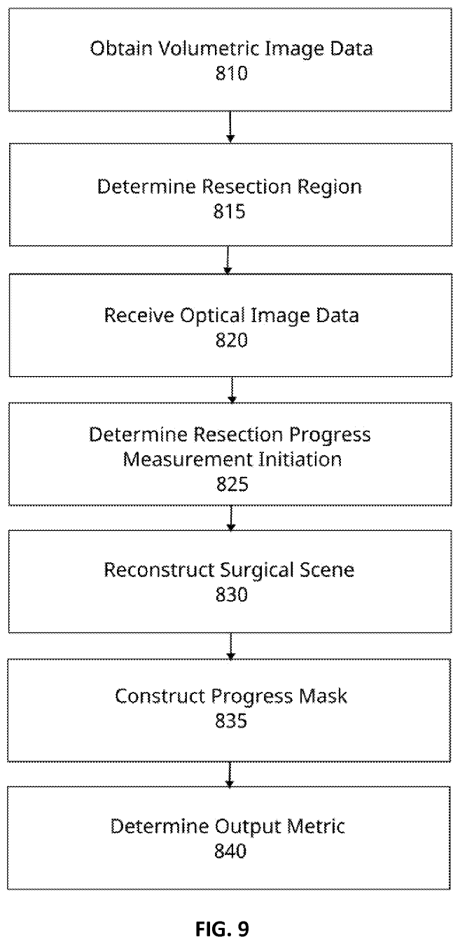

FIG. 9 shows an example computer-executed process 800 for determining resection-progress information during a surgical procedure. At step 810, volumetric image data of an anatomical region may be obtained. In some embodiments, the volumetric image data may include magnetic resonance imaging, computed tomography data, or other volumetric imaging data associated with a patient. At step 815, a resection region may be determined based on the volumetric image data. In some embodiments, the resection region may correspond to a planned resection region and may be represented using one or more masks, models, or other data structures associated with the anatomical region. At step 820, optical image data of the anatomical region may be received during the surgical procedure, such as image data captured by a surgical microscope, exoscope, or other optical imaging system.

At step 825, a determination may be made whether resection-progress measurement is to be initiated. In some embodiments, this determination may be based on one or more initiation conditions, such as whether tissue removal is underway, whether a field of view includes relevant anatomy, whether a triggering instrument is present, or whether a manual or other input has been received. At step 830, a surgical scene may be reconstructed based on the optical image data. In some embodiments, the reconstruction may include generating a depth representation, point cloud, surface representation, mesh, or other geometric representation associated with a cavity region or surrounding anatomy. At step 835, a progress mask may be constructed. In some embodiments, the progress mask may be based on comparison of a resection mask with a planned resection mask or other region representation derived from the volumetric image data. At step 840, one or more output metrics may be determined, such as a progress volume, remnant volume, progress fraction, or supra-planned resection volume. The output metric may then be used for presentation on a user interface during the surgical procedure.

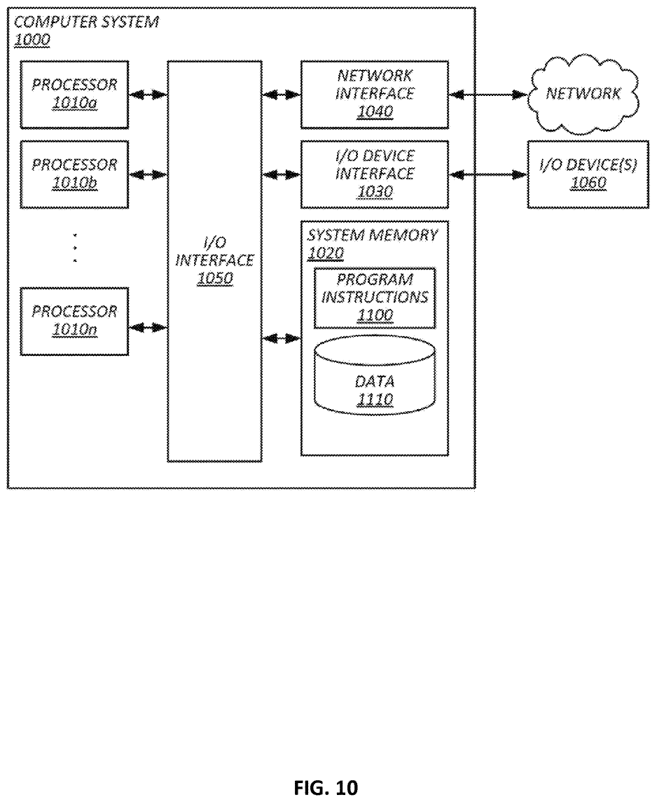

FIG. 10 is a diagram that illustrates an exemplary computing system 1000 in accordance with embodiments of the present technique. A single computing device is shown, but some embodiments of a computer system may include multiple computing devices that communicate over a network, for instance in the course of collectively executing various parts of a distributed application. Various portions of systems and methods described herein, may include or be executed on one or more computer systems similar to computing system 1000. Further, processes and modules described herein may be executed by one or more processing systems similar to that of computing system 1000.

Computing system 1000 may include one or more processors (e.g., processors 1010a-1010n) coupled to system memory 1020, an input/output I/O device interface 1030, and a network interface 1040 via an input/output (I/O) interface 1050. A processor may include a single processor or a plurality of processors (e.g., distributed processors). A processor may be any suitable processor capable of executing or otherwise performing instructions. A processor may include a central processing unit (CPU) that carries out program instructions to perform the arithmetical, logical, and input/output operations of computing system 1000. A processor may execute code (e.g., processor firmware, a protocol stack, a database management system, an operating system, or a combination thereof) that creates an execution environment for program instructions. A processor may include a programmable processor. A processor may include general or special purpose microprocessors. A processor may receive instructions and data from a memory (e.g., system memory 1020). Computing system 1000 may be a uni-processor system including one processor (e.g., processor 1010a), or a multi-processor system including any number of suitable processors (e.g., 1010a-1010n). Multiple processors may be employed to provide for parallel or sequential execution of one or more portions of the techniques described herein. Processes, such as logic flows, described herein may be performed by one or more programmable processors executing one or more computer programs to perform functions by operating on input data and generating corresponding output. Processes described herein may be performed by, and apparatus can also be implemented as, special purpose logic circuitry, e.g., an FPGA (field programmable gate array) or an ASIC (application specific integrated circuit). Computing system 1000 may include a plurality of computing devices (e.g., distributed computer systems) to implement various processing functions.

I/O device interface 1030 may provide an interface for connection of one or more I/O devices 1060 to computer system 1000. I/O devices may include devices that receive input (e.g., from a user) or output information (e.g., to a user). I/O devices 1060 may include, for example, graphical user interface presented on displays (e.g., a cathode ray tube (CRT) or liquid crystal display (LCD) monitor), pointing devices (e.g., a computer mouse or trackball), keyboards, keypads, touchpads, scanning devices, voice recognition devices, gesture recognition devices, printers, audio speakers, microphones, cameras, or the like. I/O devices 1060 may be connected to computer system 1000 through a wired or wireless connection. I/O devices 1060 may be connected to computer system 1000 from a remote location. I/O devices 1060 located on remote computer system, for example, may be connected to computer system 1000 via a network and network interface 1040.

Network interface 1040 may include a network adapter that provides for connection of computer system 1000 to a network. Network interface May 1040 may facilitate data exchange between computer system 1000 and other devices connected to the network. Network interface 1040 may support wired or wireless communication. The network may include an electronic communication network, such as the Internet, a local area network (LAN), a wide area network (WAN), a cellular communications network, or the like.

System memory 1020 may be configured to store program instructions 1100 or data 1110. Program instructions 1100 may be executable by a processor (e.g., one or more of processors 1010a-1010n) to implement one or more embodiments of the present techniques. Instructions 1100 may include modules of computer program instructions for implementing one or more techniques described herein with regard to various processing modules. Program instructions may include a computer program (which in certain forms is known as a program, software, software application, script, or code). A computer program may be written in a programming language, including compiled or interpreted languages, or declarative or procedural languages. A computer program may include a unit suitable for use in a computing environment, including as a stand-alone program, a module, a component, or a subroutine. A computer program may or may not correspond to a file in a file system. A program may be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program may be deployed to be executed on one or more computer processors located locally at one site or distributed across multiple remote sites and interconnected by a communication network.

System memory 1020 may include a tangible program carrier having program instructions stored thereon. A tangible program carrier may include a non-transitory computer readable storage medium. A non-transitory computer readable storage medium may include a machine readable storage device, a machine readable storage substrate, a memory device, or any combination thereof. Non-transitory computer readable storage medium may include non-volatile memory (e.g., flash memory, ROM, PROM, EPROM, EEPROM memory), volatile memory (e.g., random access memory (RAM), static random access memory (SRAM), synchronous dynamic RAM (SDRAM)), bulk storage memory (e.g., CD-ROM and/or DVD-ROM, hard-drives), or the like. System memory 1020 may include a non-transitory computer readable storage medium that may have program instructions stored thereon that are executable by a computer processor (e.g., one or more of processors 1010a-1010n) to cause the subject matter and the functional operations described herein. A memory (e.g., system memory 1020) may include a single memory device and/or a plurality of memory devices (e.g., distributed memory devices). Instructions or other program code to provide the functionality described herein may be stored on a tangible, non-transitory computer readable media. In some embodiments, the entire set of instructions may be stored concurrently on the media, or in some cases, different parts of the instructions may be stored on the same media at different times.

I/O interface 1050 may be configured to coordinate I/O traffic between processors 1010a-1010n, system memory 1020, network interface 1040, I/O devices 1060, and/or other peripheral devices. I/O interface 1050 may perform protocol, timing, or other data transformations to convert data signals from one component (e.g., system memory 1020) into a format suitable for use by another component (e.g., processors 1010a-1010n). I/O interface 1050 may include support for devices attached through various types of peripheral buses, such as a variant of the Peripheral Component Interconnect (PCI) bus standard or the Universal Serial Bus (USB) standard.

Embodiments of the techniques described herein may be implemented using a single instance of computer system 1000 or multiple computer systems 1000 configured to host different portions or instances of embodiments. Multiple computer systems 1000 may provide for parallel or sequential processing/execution of one or more portions of the techniques described herein.

Those skilled in the art will appreciate that computer system 1000 is merely illustrative and is not intended to limit the scope of the techniques described herein. Computer system 1000 may include any combination of devices or software that may perform or otherwise provide for the performance of the techniques described herein. For example, computer system 1000 may include or be a combination of a cloud-computing system, a data center, a server rack, a server, a virtual server, a desktop computer, a laptop computer, a tablet computer, a server device, a client device, a mobile telephone, a personal digital assistant (PDA), a mobile audio or video player, a game console, a vehicle-mounted computer, or a Global Positioning System (GPS), or the like. Computer system 1000 may also be connected to other devices that are not illustrated, or may operate as a stand-alone system. In addition, the functionality provided by the illustrated components may in some embodiments be combined in fewer components or distributed in additional components. Similarly, in some embodiments, the functionality of some of the illustrated components may not be provided or other additional functionality may be available.

Those skilled in the art will also appreciate that while various items are illustrated as being stored in memory or on storage while being used, these items or portions of them may be transferred between memory and other storage devices for purposes of memory management and data integrity. Alternatively, in other embodiments some or all of the software components may execute in memory on another device and communicate with the illustrated computer system via inter-computer communication. Some or all of the system components or data structures may also be stored (e.g., as instructions or structured data) on a computer-accessible medium or a portable article to be read by an appropriate drive, various examples of which are described above. In some embodiments, instructions stored on a computer-accessible medium separate from computer system 1000 may be transmitted to computer system 1000 via transmission media or signals such as electrical, electromagnetic, or digital signals, conveyed via a communication medium such as a network or a wireless link. Various embodiments may further include receiving, sending, or storing instructions or data implemented in accordance with the foregoing description upon a computer-accessible medium. Accordingly, the present techniques may be practiced with other computer system configurations.

In block diagrams, illustrated components are depicted as discrete functional blocks, but embodiments are not limited to systems in which the functionality described herein is organized as illustrated. The functionality provided by each of the components may be provided by software or hardware modules that are differently organized than is presently depicted, for example such software or hardware may be intermingled, conjoined, replicated, broken up, distributed (e.g., within a data center or geographically), or otherwise differently organized. The functionality described herein may be provided by one or more processors of one or more computers executing code stored on a tangible, non-transitory, machine readable medium. In some embodiments, notwithstanding use of the singular term “medium,” the instructions may be distributed on different storage devices associated with different computing devices, for instance, with each computing device having a different subset of the instructions, an embodiment consistent with usage of the singular term “medium” herein. In some embodiments, third party content delivery networks may host some or all of the information conveyed over networks, in which case, to the extent information (e.g., content) is said to be supplied or otherwise provided, the information may be provided by sending instructions to retrieve that information from a content delivery network.

The reader should appreciate that the present application describes several independently useful techniques. Rather than separating those techniques into multiple isolated patent applications, applicants have grouped these techniques into a single document because their related subject matter lends itself to economies in the application process. But the distinct advantages and aspects of such techniques should not be conflated. In some embodiments, embodiments address all of the deficiencies noted herein, but it should be understood that the techniques are independently useful, and some embodiments address only a subset of such problems or offer other, unmentioned benefits that will be apparent to those of skill in the art reviewing the present disclosure. Due to cost constraints, some techniques disclosed herein may not be presently claimed and may be claimed in later filings, such as continuation applications or by amending the present claims. Similarly, due to space constraints, neither the Abstract nor the Summary of the Invention sections of the present document should be taken as containing a comprehensive listing of all such techniques or all aspects of such techniques.

It should be understood that the description and the drawings are not intended to limit the present techniques to the particular form disclosed, but to the contrary, the intention is to cover all modifications, equivalents, and alternatives falling within the spirit and scope of the present techniques as defined by the appended claims. Further modifications and alternative embodiments of various aspects of the techniques will be apparent to those skilled in the art in view of this description. Accordingly, this description and the drawings are to be construed as illustrative only and are for the purpose of teaching those skilled in the art the general manner of carrying out the present techniques. It is to be understood that the forms of the present techniques shown and described herein are to be taken as examples of embodiments. Elements and materials may be substituted for those illustrated and described herein, parts and processes may be reversed or omitted, and certain features of the present techniques may be utilized independently, all as would be apparent to one skilled in the art after having the benefit of this description of the present techniques. Changes may be made in the elements described herein without departing from the spirit and scope of the present techniques as described in the following claims. Headings used herein are for organizational purposes only and are not meant to be used to limit the scope of the description.

As used throughout this application, the word “may” is used in a permissive sense (i.e., meaning having the potential to), rather than the mandatory sense (i.e., meaning must). The words “include”, “including”, and “includes” and the like mean including, but not limited to. As used throughout this application, the singular forms “a,” “an,” and “the” include plural referents unless the content explicitly indicates otherwise. Thus, for example, reference to “an element” or “a element” includes a combination of two or more elements, notwithstanding use of other terms and phrases for one or more elements, such as “one or more.” The term “or” is, unless indicated otherwise, non-exclusive, i.e., encompassing both “and” and “or.” Terms describing conditional relationships, e.g., “in response to X, Y,” “upon X, Y,”, “if X, Y,” “when X, Y,” and the like, encompass causal relationships in which the antecedent is a necessary causal condition, the antecedent is a sufficient causal condition, or the antecedent is a contributory causal condition of the consequent, e.g., “state X occurs upon condition Y obtaining” is generic to “X occurs solely upon Y” and “X occurs upon Y and Z.” Such conditional relationships are not limited to consequences that instantly follow the antecedent obtaining, as some consequences may be delayed, and in conditional statements, antecedents are connected to their consequents, e.g., the antecedent is relevant to the likelihood of the consequent occurring. Statements in which a plurality of attributes or functions are mapped to a plurality of objects (e.g., one or more processors performing steps A, B, C, and D) encompasses both all such attributes or functions being mapped to all such objects and subsets of the attributes or functions being mapped to subsets of the attributes or functions (e.g., both all processors each performing steps A-D, and a case in which processor 1 performs step A, processor 2 performs step B and part of step C, and processor 3 performs part of step C and step D), unless otherwise indicated. Similarly, reference to “a computer system” performing step A and “the computer system” performing step B can include the same computing device within the computer system performing both steps or different computing devices within the computer system performing steps A and B. Further, unless otherwise indicated, statements that one value or action is “based on” another condition or value encompass both instances in which the condition or value is the sole factor and instances in which the condition or value is one factor among a plurality of factors. Unless otherwise indicated, statements that “each” instance of some collection have some property should not be read to exclude cases where some otherwise identical or similar members of a larger collection do not have the property, i.e., each does not necessarily mean each and every. Limitations as to sequence of recited steps should not be read into the claims unless explicitly specified, e.g., with explicit language like “after performing X, performing Y,” in contrast to statements that might be improperly argued to imply sequence limitations, like “performing X on items, performing Y on the X′ed items,” used for purposes of making claims more readable rather than specifying sequence. Statements referring to “at least Z of A, B, and C,” and the like (e.g., “at least Z of A, B, or C”), refer to at least Z of the listed categories (A, B, and C) and do not require at least Z units in each category. Unless specifically stated otherwise, as apparent from the discussion, it is appreciated that throughout this specification discussions utilizing terms such as “processing,” “computing,” “calculating,” “determining” or the like refer to actions or processes of a specific apparatus, such as a special purpose computer or a similar special purpose electronic processing/computing device. Features described with reference to geometric constructs, like “parallel,” “perpendicular/orthogonal,” “square”, “cylindrical,” and the like, should be construed as encompassing items that substantially embody the properties of the geometric construct, e.g., reference to “parallel” surfaces encompasses substantially parallel surfaces. The permitted range of deviation from Platonic ideals of these geometric constructs is to be determined with reference to ranges in the specification, and where such ranges are not stated, with reference to industry norms in the field of use, and where such ranges are not defined, with reference to industry norms in the field of manufacturing of the designated feature, and where such ranges are not defined, features substantially embodying a geometric construct should be construed to include those features within 15% of the defining attributes of that geometric construct. The terms “first”, “second”, “third,” “given” and so on, if used in the claims, are used to distinguish or otherwise identify, and not to show a sequential or numerical limitation. As is the case in ordinary usage in the field, data structures and formats described with reference to uses salient to a human need not be presented in a human-intelligible format to constitute the described data structure or format, e.g., text need not be rendered or even encoded in Unicode or ASCII to constitute text; images, maps, and data-visualizations need not be displayed or decoded to constitute images, maps, and data-visualizations, respectively; speech, music, and other audio need not be emitted through a speaker or decoded to constitute speech, music, or other audio, respectively. Computer implemented instructions, commands, and the like are not limited to executable code and can be implemented in the form of data that causes functionality to be invoked, e.g., in the form of arguments of a function or API call. To the extent bespoke noun phrases (and other coined terms) are used in the claims and lack a self-evident construction, the definition of such phrases may be recited in the claim itself, in which case, the use of such bespoke noun phrases should not be taken as invitation to impart additional limitations by looking to the specification or extrinsic evidence.

In this patent, to the extent any U.S. patents, U.S. patent applications, or other materials (e.g., articles) have been incorporated by reference, the text of such materials is only incorporated by reference to the extent that no conflict exists between such material and the statements and drawings set forth herein. In the event of such conflict, the text of the present document governs, and terms in this document should not be given a narrower reading in virtue of the way in which those terms are used in other materials incorporated by reference.

The present techniques will be better understood with reference to the following enumerated embodiments:
1. A tangible, non-transitory, machine-readable medium storing instructions that when executed by one or more physical processors cause the one or more physical processors to perform operations comprising: obtaining, with a computer system, a first volumetric image dataset of an anatomical region of a patient imaged before a surgical procedure; obtaining, with the computer system, a planned resection model associated with the first volumetric image dataset, the planned resection model indicating a surgeon-planned resection region within the anatomical region; obtaining, with the computer system, optical image data of the anatomical region captured during the surgical procedure; obtaining, with the computer system, a signal indicating that a resection progress measurement is to be initiated; in response to obtaining the signal, constructing, with the computer system, based on the optical image data, a three-dimensional mesh reconstruction of tissue visible in the anatomical region during the surgical procedure from the optical image data; determining, with the computer system, an updated anatomical model using the first volumetric image dataset, the three-dimensional mesh reconstruction, and a model configured to update the first volumetric image dataset to account for intraoperative tissue displacement in the anatomical region; comparing, with the computer system, the updated anatomical model with the planned resection model to determine a resection progress indication relative to the surgeon-planned resection region; and causing, with the computer system, via a user interface, presentation of output indicating resection progress, the output identifying at least one of a portion of the surgeon-planned resection region already resected or a portion remaining to be resected.
2. The medium of embodiment 1, wherein the received signal is generated based on at least one of: a determination that tissue removal from the anatomical region is underway, a determination that a field of view of the optical image data contains anatomy associated with the anatomical region, a determination that a duration of time since a previous measurement has elapsed, detection of a triggering instrument or an input command, or receipt of a manual trigger.
3. The medium of embodiment 1, wherein receiving the optical image data comprises receiving, from a stereoscopic camera system located in an operating room, first image data from a first optical channel and second image data from a second optical channel, the first image data and the second image data capturing the anatomical region during the surgical procedure from different viewpoints.
4. The medium of embodiment 1, further comprising: prior to constructing the three-dimensional mesh reconstruction, evaluating the optical image data to determine whether the optical image data satisfies one or more measurement quality conditions including at least one of a visibility condition, a focus condition, a motion condition, or an obstruction condition, wherein the three-dimensional mesh reconstruction is performed in response to determining that the one or more measurement quality conditions are satisfied.
5. The medium of embodiment 1, wherein the planned resection region extends beyond a boundary of a lesion in a first portion of the anatomical region and remains within the boundary of the lesion in a second portion of the anatomical region.
6. A tangible, non-transitory, machine-readable medium storing instructions that when executed by a data processing apparatus cause the data processing apparatus to perform operations comprising: obtaining, with a computer system, a first volumetric image dataset of an anatomical region of a patient; obtaining, with the computer system, a plan mask based on the first volumetric image dataset and corresponding to a planned resection region that includes at least a portion of a lesion within the anatomical region; obtaining, with the computer system, an organ region mask based on the first volumetric image dataset and corresponding to the anatomical region; receiving, with the computer system, image data of the anatomical region captured during a surgical procedure; determining, with the computer system, that a resection progress measurement is to be initiated; in response to determining that the resection progress measurement is to be initiated, performing, with the computer system, a three-dimensional reconstruction of a surgical scene represented in the image data and determining a surgical surface representation by: determining from the image data, a surgical segmentation corresponding to removed tissue in the anatomical region; mapping the surgical segmentation to the three-dimensional reconstruction to determine a surgical region; and transforming coordinates of the surgical region from a coordinate system of the three-dimensional reconstruction into the coordinate system of the first volumetric image dataset; constructing, with the computer system, from the surgical surface representation, a resection mask; constructing, with the computer system, at least one of the following: a progress mask corresponding to an intersection of the resection mask and the plan mask, a remnant mask corresponding to a portion of the plan mask outside the resection mask, or a supra-planned mask corresponding to a portion of the resection mask outside the plan mask and within the organ region mask; and determining, with the computer system, at least one output metric indicative of at least one of the following: a progress volume determined based on the progress mask, a remnant volume determined based on the remnant mask, or a supra-planned resection volume based on the supra-planned mask.
7. The medium of embodiment 6, the operations comprising: constructing the progress mask, the remnant mask, and the supra-planned mask; and determining the progress volume, the remnant volume, and the supra-planned resection volume.
8. The medium of embodiment 6, wherein determining that a resection progress measurement is to be initiated is based on at least one of: a determination that tissue removal from the anatomical region is underway, a determination that a field of view of the image data contains anatomy associated with the anatomical region, detection of a triggering instrument or an input command, or receipt of a manual trigger.
9. The medium of embodiment 6, wherein the surgical surface representation comprises at least one of a depth map, a point cloud, or a surface mesh.
10. The medium of embodiment 6, wherein constructing a resection mask comprises: determining a surface mesh corresponding to the surgical region; determining a plurality of boundary loops in the surface mesh; ranking the boundary loops by length; closing one or more boundary loops other than a largest boundary loop; forming a cap from the largest boundary loop by extruding the largest boundary loop to form a cap rim, closing the cap rim, and filling a space between the cap rim and the largest boundary loop with faces to form a watertight mesh; and converting the watertight mesh into a resection mask on a voxel grid of the first volumetric image dataset.
11. The medium of embodiment 6, wherein constructing the progress mask, remnant mask, and supra-planned mask is based on Boolean operations involving the resection mask, the plan mask, and the organ region mask.
12. The medium of embodiment 6, further comprising: causing presentation of the at least one output metric on a user interface comprising: a graphical representation of the anatomical region based on at least one of the image data, the three-dimensional reconstruction, or the first volumetric image dataset; and a visual indication of the at least one output metric comprising at least one of the progress volume, the remnant volume, the progress fraction, or the supra-planned resection volume.
13. The medium of embodiment 12, wherein causing presentation of the at least one output metric comprises causing presentation of: a first indication of an amount of tissue removed within the planned resection region; a second indication of an amount of tissue remaining within the planned resection region; and a third indication of an amount of tissue removed outside the planned resection region and within the organ region mask, wherein the first indication, the second indication, and the third indication are presented concurrently on the user interface.
14. The medium of embodiment 6, wherein the first volumetric image dataset is acquired prior to the surgical procedure.
15. The medium of embodiment 6, further comprising: prior to performing the three-dimensional reconstruction, evaluating the image data to determine whether the image data satisfies one or more measurement quality conditions including at least one of a visibility condition, a focus condition, a motion condition, or an obstruction condition, wherein the three-dimensional reconstruction is performed in response to determining that the one or more measurement quality conditions are satisfied.
16. The medium of embodiment 6, wherein obtaining the plan mask comprises receiving surgeon input specifying at least a portion of the planned resection region.
17. The medium of embodiment 6, wherein receiving the image data comprises receiving, from a stereoscopic camera system located in an operating room, first image data from a first optical channel and second image data from a second optical channel, the first image data and the second image data capturing the anatomical region during the surgical procedure from different viewpoints.
18. The medium of embodiment 6, wherein the anatomical region comprises brain tissue, the planned resection region corresponds to tissue to be removed during a neurosurgical procedure, and the image data is captured during the neurosurgical procedure.
19. The medium of embodiment 6, wherein constructing the resection mask comprises steps for constructing the resection mask.
20. The medium of embodiment 6, wherein transforming coordinates of the surgical region from the coordinate system of the three-dimensional reconstruction into the coordinate system of the first volumetric image dataset comprises: determining a surface mesh corresponding to the surgical region in camera-relative coordinates; mapping the surface mesh from the camera-relative coordinates to scanner coordinates of the first volumetric image dataset; and converting the surface mesh from the scanner coordinates into voxel coordinates of the first volumetric image dataset using an inverse affine transformation.
21. The medium of embodiment 6, wherein the image data of the anatomical region captured during a surgical procedure comprises optical image data captured with a plurality of cameras.
22. The medium of embodiment 6, wherein the image data of the anatomical region captured during a surgical procedure comprises ultrasound data.
23. The medium of embodiment 6, wherein performing the three-dimensional reconstruction of the surgical scene represented in the image data comprises: forming a mesh model of the surgical scene; forming a point cloud of the surgical scene; forming a Gaussian splat of the surgical scene; or forming a neural radiance field of the surgical scene.
24. The medium of embodiment 6, wherein constructing the resection mask from the cavity surface representation comprises: determining a surface mesh corresponding to the cavity region; determining one or more boundary loops in the surface mesh and closing the one or more boundary loops to form a watertight mesh corresponding to tissue determined to have been removed; converting positions of vertices of the watertight mesh from scanner-space coordinates into voxel-space coordinates of the first volumetric image dataset using an inverse affine transformation associated with the first volumetric image dataset; determining a voxel shell corresponding to voxels intersected by the watertight mesh on a voxel grid of the first volumetric image dataset; and filling an interior of the voxel shell to determine the resection mask on the voxel grid of the first volumetric image dataset.
25. A method, comprising the operations of any one of embodiments 1-24.
26. A computer system comprising: one or more processors; and memory storing instructions that when executed by at least some of the processors effectuate the operations of any one of the embodiments 1-24.

1. A tangible, non-transitory, machine-readable medium storing instructions that when executed by one or more physical processors cause the one or more physical processors to perform operations comprising:
obtaining, with a computer system, a volumetric image dataset of an anatomical region of a patient imaged before a surgical procedure;
obtaining, with the computer system, a planned resection model associated with the volumetric image dataset, the planned resection model indicating a surgeon-planned resection region within the anatomical region;
obtaining, with the computer system, optical image data of the anatomical region captured during the surgical procedure;
obtaining, with the computer system, a signal indicating that a resection progress measurement is to be initiated;
in response to obtaining the signal, constructing, with the computer system, based on the optical image data, a three-dimensional mesh reconstruction of tissue visible in the anatomical region during the surgical procedure from the optical image data;
determining, with the computer system, an updated anatomical model using the first volumetric image dataset, the three-dimensional mesh reconstruction, and a model configured to update the volumetric image dataset to account for intraoperative tissue displacement in the anatomical region;
comparing, with the computer system, the updated anatomical model with the planned resection model to determine a resection progress indication relative to the surgeon-planned resection region; and
causing, with the computer system, via a user interface, presentation of output indicating resection progress, the output identifying at least one of a portion of the surgeon-planned resection region already resected or a portion remaining to be resected, wherein the planned resection region extends beyond a boundary of a lesion in a first portion of the anatomical region and remains within the boundary of the lesion in a second portion of the anatomical region.

2. The medium of claim 1, wherein the obtained signal is generated based on at least one of:
a determination that tissue removal from the anatomical region is underway, a determination that a field of view of the optical image data contains anatomy associated with the anatomical region,
a determination that a duration of time since a previous measurement has elapsed,
detection of a triggering instrument or an input command, or
receipt of a manual trigger.

3. The medium of claim 1, wherein obtaining the optical image data comprises receiving, from a stereoscopic camera system located in an operating room, first image data from a first optical channel and second image data from a second optical channel, the first image data and the second image data capturing the anatomical region during the surgical procedure from different viewpoints.

4. The medium of claim 1, further comprising:
prior to constructing the three-dimensional mesh reconstruction, evaluating the optical image data to determine whether the optical image data satisfies one or more measurement quality conditions including at least one of a visibility condition, a focus condition, a motion condition, or an obstruction condition,
wherein the three-dimensional mesh reconstruction is performed in response to determining that the one or more measurement quality conditions are satisfied.

5. (canceled)

6. A tangible, non-transitory, machine-readable medium storing instructions that when executed by a data processing apparatus cause the data processing apparatus to perform operations comprising:
obtaining, with a computer system, a volumetric image dataset of an anatomical region of a patient;
obtaining, with the computer system, a plan mask based on the volumetric image dataset and corresponding to a planned resection region that includes at least a portion of a lesion within the anatomical region;
obtaining, with the computer system, an organ region mask based on the volumetric image dataset and corresponding to the anatomical region;
receiving, with the computer system, image data of the anatomical region captured during a surgical procedure;
determining, with the computer system, that a resection progress measurement is to be initiated;
in response to determining that the resection progress measurement is to be initiated, performing, with the computer system, a three-dimensional reconstruction of a surgical scene represented in the image data and determining a surgical surface representation by:
determining from the image data, a surgical segmentation including removed tissue in the anatomical region;
mapping the surgical segmentation to the three-dimensional reconstruction to determine a surgical region; and
transforming coordinates of the surgical region from a coordinate system of the three-dimensional reconstruction into the coordinate system of the volumetric image dataset;
constructing, with the computer system, from the surgical surface representation, a resection mask;
constructing, with the computer system, at least one of the following:
a progress mask corresponding to an intersection of the resection mask and the plan mask,
a remnant mask corresponding to a portion of the plan mask outside the resection mask, or
a supra-planned mask corresponding to a portion of the resection mask outside the plan mask and within the organ region mask; and
determining, with the computer system, at least one output metric indicative of at least one of the following:
a progress volume determined based on the progress mask,
a remnant volume determined based on the remnant mask, or
a supra-planned resection volume based on the supra-planned mask.

7. The medium of claim 6, the operations comprising:
constructing the progress mask, the remnant mask, and the supra-planned mask; and
determining the progress volume, the remnant volume, and the supra-planned resection volume.

8. The medium of claim 6, wherein determining that the resection progress measurement is to be initiated is based on at least one of:
a determination that tissue removal from the anatomical region is underway,
a determination that a field of view of the image data contains anatomy associated with the anatomical region,
detection of a triggering instrument or an input command, or
receipt of a manual trigger.

9. The medium of claim 6, wherein the surgical surface representation comprises at least one of a depth map, a point cloud, or a surface mesh.

10. The medium of claim 6, wherein constructing the resection mask comprises:
determining a surface mesh corresponding to the surgical region;
determining a plurality of boundary loops in the surface mesh;
ranking the boundary loops by length;
closing one or more boundary loops other than a largest boundary loop;
forming a cap from the largest boundary loop by extruding the largest boundary loop to form a cap rim, closing the cap rim, and filling a space between the cap rim and the largest boundary loop with faces to form a watertight mesh; and
converting the watertight mesh into the resection mask on a voxel grid of the volumetric image dataset.

11. The medium of claim 6, wherein constructing the progress mask, remnant mask, and supra-planned mask is based on Boolean operations involving the resection mask, the plan mask, and the organ region mask.

12. The medium of claim 6, further comprising:
causing presentation of the at least one output metric on a user interface comprising:
a graphical representation of the anatomical region based on at least one of the image data, the three-dimensional reconstruction, or the volumetric image dataset; and
a visual indication of the at least one output metric comprising at least one of the progress volume, the remnant volume, a progress fraction, or the supra-planned resection volume.

13. The medium of claim 12, wherein causing presentation of the at least one output metric comprises causing presentation of:
a first indication of an amount of tissue removed within the planned resection region;
a second indication of an amount of tissue remaining within the planned resection region; and
a third indication of an amount of tissue removed outside the planned resection region and within the organ region mask,
wherein the first indication, the second indication, and the third indication are presented concurrently on the user interface.

14. The medium of claim 6, wherein the volumetric image dataset is acquired prior to the surgical procedure.

15. The medium of claim 6, further comprising:
prior to performing the three-dimensional reconstruction, evaluating the image data to determine whether the image data satisfies one or more measurement quality conditions including at least one of a visibility condition, a focus condition, a motion condition, or an obstruction condition,
wherein the three-dimensional reconstruction is performed in response to determining that the one or more measurement quality conditions are satisfied.

16. The medium of claim 6, wherein obtaining the plan mask comprises receiving surgeon input specifying at least a portion of the planned resection region.

17. The medium of claim 6, wherein receiving the image data comprises receiving, from a stereoscopic camera system located in an operating room, first image data from a first optical channel and second image data from a second optical channel, the first image data and the second image data capturing the anatomical region during the surgical procedure from different viewpoints.

18. The medium of claim 6, wherein the anatomical region comprises brain tissue, the planned resection region corresponds to tissue to be removed during a neurosurgical procedure, and the image data is captured during the neurosurgical procedure.

19. The medium of claim 6, wherein constructing the resection mask comprises steps for constructing the resection mask.

20. The medium of claim 6, wherein transforming coordinates of the surgical region from the coordinate system of the three-dimensional reconstruction into the coordinate system of the volumetric image dataset comprises:
determining a surface mesh corresponding to the surgical region in camera-relative coordinates;
mapping the surface mesh from the camera-relative coordinates to scanner coordinates of the volumetric image dataset; and
converting the surface mesh from the scanner coordinates into voxel coordinates of the volumetric image dataset using an inverse affine transformation.

21. The medium of claim 6, wherein the image data of the anatomical region captured during the surgical procedure comprises optical image data captured with a plurality of cameras.

22. The medium of claim 6, wherein the image data of the anatomical region captured during the surgical procedure comprises ultrasound data.

23. The medium of claim 6, wherein performing the three-dimensional reconstruction of the surgical scene represented in the image data comprises:
forming a mesh model of the surgical scene;
forming a point cloud of the surgical scene;
forming a Gaussian splat of the surgical scene; or
forming a neural radiance field of the surgical scene.

24. The medium of claim 6, wherein constructing the resection mask comprises:
determining a surface mesh corresponding to a cavity region;
determining one or more boundary loops in the surface mesh and closing the one or more boundary loops to form a watertight mesh corresponding to tissue determined to have been removed; converting positions of vertices of the watertight mesh from scanner-space coordinates into voxel-space coordinates of the volumetric image dataset using an inverse affine transformation associated with the volumetric image dataset;
determining a voxel shell corresponding to voxels intersected by the watertight mesh on a voxel grid of the volumetric image dataset; and
filling an interior of the voxel shell to determine the resection mask on the voxel grid of the volumetric image dataset.

25. A method, comprising:
obtaining, with a computer system, a volumetric image dataset of an anatomical region of a patient;
obtaining, with the computer system, a plan mask based on the volumetric image dataset and corresponding to a planned resection region that includes at least a portion of a lesion within the anatomical region;
obtaining, with the computer system, an organ region mask based on the volumetric image dataset and corresponding to the anatomical region;
receiving, with the computer system, image data of the anatomical region captured during a surgical procedure;
determining, with the computer system, that a resection progress measurement is to be initiated;
in response to determining that the resection progress measurement is to be initiated, performing, with the computer system, a three-dimensional reconstruction of a surgical scene represented in the image data and determining a surgical surface representation by:
determining from the image data, a surgical segmentation including removed tissue in the anatomical region;
mapping the surgical segmentation to the three-dimensional reconstruction to determine a surgical region; and
transforming coordinates of the surgical region from a coordinate system of the three-dimensional reconstruction into the coordinate system of the volumetric image dataset;
constructing, with the computer system, from the surgical surface representation, a resection mask;
constructing, with the computer system, at least one of the following:
a progress mask corresponding to an intersection of the resection mask and the plan mask,
a remnant mask corresponding to a portion of the plan mask outside the resection mask, or
a supra-planned mask corresponding to a portion of the resection mask outside the plan mask and within the organ region mask; and
determining, with the computer system, at least one output metric indicative of at least one of the following:
a progress volume determined based on the progress mask,
a remnant volume determined based on the remnant mask, or
a supra-planned resection volume based on the supra-planned mask.
